(19) United States
(12) Patent Application Publication
(10) Pub. No.: US 2025/0115617 A1
(43) Pub. Date: Apr. 10, 2025

(54) MACROCYCLIC CYCLOPHANES

(71) Applicant: Iowa State University Research Foundation, Inc., Ames, IA (US)

(72) Inventors: Junqi Li, Ames, IA (US); Shengkai Wei, Ames, IA (US); Liang-Yu Chen, Ames, IA (US)

(21) Appl. No.: 18/891,879

(22) Filed: Sep. 20, 2024

Related U.S. Application Data
(60) Provisional application No. 63/586,784, filed on Sep. 29, 2023.

(51) Int. Cl. C07D 491/08; B01J 23/44

(52) U.S. Cl. CPC C07D 491/08; B01J 23/44

(57) ABSTRACT

The disclosure provides a method of preparing chiral metacyclophanes. The method involves palladium-catalyzed cross-coupling of a meta-substituted aryl group with base and a chiral ligand. The disclosure also provides metacyclophane compounds, which include diaryl and monoaryl macrocycles.

### CROSS-REFERENCE TO RELATED APPLICATIONS

This application claims the benefit of U.S. Provisional Application No. 63/586,784, filed Sep. 29, 2023, entitled “MACROCYCLIC CYCLOPHANES,” which is incorporated herein by reference in its entirety.

### BACKGROUND

Cyclophanes have unique structural and electronic properties that make them useful in various fields of chemistry and materials science. While few cyclophanes exist in nature, these molecules can exhibit highly selective binding and distinctive host-guest interactions. Cyclophanes can exhibit various forms of steroisomerism, including point chirality and planar chirality. Chiral cyclophanes are of particular interest due to the unique binding properties of the macrocyclic structure and the ability to introduce chirality in host-guest contexts, supramolecular interactions, and ligand interactions. Chiral cyclophanes also represent a diverse pharmocophore for drug discovery, which can be useful as either a structural or functional element. Despite the utility of planar chiral compounds in a wide variety of settings, there remains a need for new catalytic enantioselective methods for synthesizing cyclophanes and installing planar chirality.

### SUMMARY OF THE INVENTION

The present disclosure provides a method of synthesizing a chiral metacyclophane, comprising contacting a substrate with a palladium cross-coupling catalyst, base, and a chiral ligand, wherein the substrate has a structure according to Formula I, wherein Q is CH or N; W is CH or N; X is a halide; Y is a sterically bulky group; m is 0 to 10; n is 0 to 10; and Z is a divalent linking group.

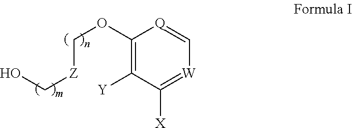

The present disclosure provides a compound having a structure according to Formula II, or a salt thereof, wherein Q is CH or N; W is CH or N; Y is a sterically bulky group; m is 0 to 10; n is 0 to 10; and Z is a divalent linking group.

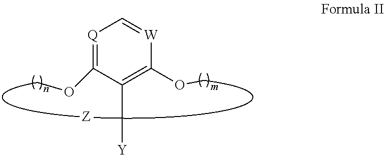

The present disclosure provides a compound having a structure according to Formula III, or a salt thereof, wherein Y is a sterically bulky group; m is 0 to 10; n is 0 to 10; and Z is a divalent linking group.

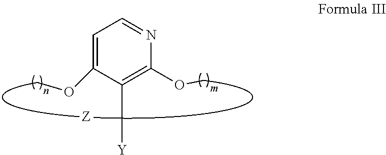

The present disclosure provides enantioselective macrocyclizations that permit access to structurally diverse planar chiral metacyclophanes. A planar chiral metacyclophane represent a potential source of chemically diverse chiral molecules that do not necessarily derive chirality from a sp3-hybridized carbon center. Planar chirality derives from the molecule as a whole, but control of molecular configuration is notoriously difficult. Moreover, macrocyclizations can be difficult to predict. Many techniques that work for non-macrocyclic ring closures, or that work for intermolecular reactions, do not work in the context of macrocyclization. The presently described synthetic method surprising imparts chirality onto macrocyclized cyclophanes.

### BRIEF DESCRIPTION OF FIGURES

FIG. 1 illustrates planar chirality in cyclophanes.

FIG. 2 illustrates a syn conformation of an (Rp)-enantiomer, based on x-ray crystal data.

FIG. 3 provides NMR data illustrating how conformation was derived.

FIG. 4 illustrates how position of the nitrogen in the starting substrate can predict entantioselection of the resulting cyclophane.

### DETAILED DESCRIPTION OF THE INVENTION

The present disclosure provides methods useful for obtaining macrocyclic cyclophanes, including planar chiral cyclophanes. The present disclosure also provides various macrocyclic cyclophane compounds.

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀) hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)—CH(CH₃)(CH₂CH₃), —CH₂CH₂—CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂—CH(CH₃)₂, —CH(CH₃)CH(CH₃)CH(CH₃)₂, —CH(CH₂CH₃)CH(CH₃)CH(CH₃)(CH₂CH₃), and others. Further examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups. A divalent “alkyl” may also be referred to as an alkylene, e.g., methylene and ethylene.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di-or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof. A divalent “aryl” group may also be referred to as an arylene, e.g., a 1,4-phenylene, a 1,3-phenylene, and a 1,2-phenylene.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro -benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “monovalent” as used herein refers to a substituent having a single point of attachment. The term “divalent” as used herein refers to a substituent having two points of attachment. For example, an unsubstituted monovalent phenyl group is “—C₆H₅” while an unsubstituted divalent phenyl (or phenylene) group is “—C₆H₄—”.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b}) hydrocarbyl, wherein a and b are integers and mean having any of a to b number of carbon atoms. For example, (C₁-C₄) hydrocarbyl means the hydrocarbyl group can be methyl (C₁), ethyl (C₂), propyl (C₃), or butyl (C₄), and (C₀-C_{b}) hydrocarbyl means in certain embodiments there is no hydrocarbyl group.

The term “solvent” as used herein refers to a liquid that can dissolve a solid, liquid, or gas. Non-limiting examples of solvents are silicones, organic compounds, water, alcohols, ionic liquids, and supercritical fluids. Examples of organic solvents are THF, diethyl ether, benzene, toluene, dichloromethane, hexane, acetone, acetonitrile, ethanol, formaldehyde, chloroform, carbon tetrachloride, and acetic acid. The present conducted reactions may be conducted in a solvent. In other embodiments, the reaction may be conducted neat.

The term “room temperature” as used herein refers to a temperature of about 15° C. to 28° C.

The term “standard temperature and pressure” as used herein refers to about 20° C. and about 101 kPa.

In various aspects, the compounds of the present disclosure can be formulated as salts. The salts can include pharmaceutically acceptable salts. The salts can include acid salts having a positively charged counterion can include any suitable positively charged counterion. For example, the counterion can be boron tetrafluoride (BF₄⁺), ammonium (NH₄⁺), or an alkali metal such as sodium (Na⁺), potassium (K⁺), or lithium (Li⁺). In some embodiments, the counterion can have a positive charge greater than +1, which can in some embodiments complex to multiple ionized groups, such as Zn²⁺, Al³⁺, or alkaline earth metals such as Ca²⁺ or Mg²⁺. In further examples, the salts can be acid or metal salts, e.g., alkali or alkali earth salts. Salts can be prepared in situ during the final isolation and purification of the compound by separately reacting the base or acid functions in the compound with a suitable organic or inorganic acid or base, respectively. Representative salts include, but are not limited to, acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-naphthalene-sulfonate, oxalate, pamoate, pectinate, persulfate, 3-phenylproionate, picrate, pivalate, propionate, succinate, sulfate, tartrate, thiocyanate, p-toluenesulfonate and undecanoate. Also, the basic nitrogen-containing groups can be quaternized with such agents as alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. In various aspects, salts having a negatively charged counterion can include any suitable negatively charged counterion. For example, the counterion can be boron tetrafluoride (BF₄⁻), perchlorate (ClO₄⁻), sulfate, carbonate, bicarbonate, chloride, bromide, iodide, nitrate, and triflate.

### Synthesis of Macrocyclic Cyclophanes

The present disclosure provides a method of synthesizing macrocyclic cyclophanes, including planar chiral macrocyclic cyclophanes. Cyclophanes are a class of organic compounds composed of an aromatic ring (e.g., phenyl or pyridine) separated by a chain of atoms forming a bridge between two non-adjacent positions of the aromatic ring, leading to a cyclic structure. The resulting structure provides a ring in which annulation can extend out of the plane of the aromatic ring. The aromatic ring in cyclophanes can exhibit significant strain and distortion. The resulting compounds are desirable components in pharmaceuticals, host-guest agents, supramolecular assemblies, sensors, catalysis, and ligand. The present approach permits access to enantiomerically enriched cyclophanes, which are particularly to impart chiral selectivity into the aforementioned uses.

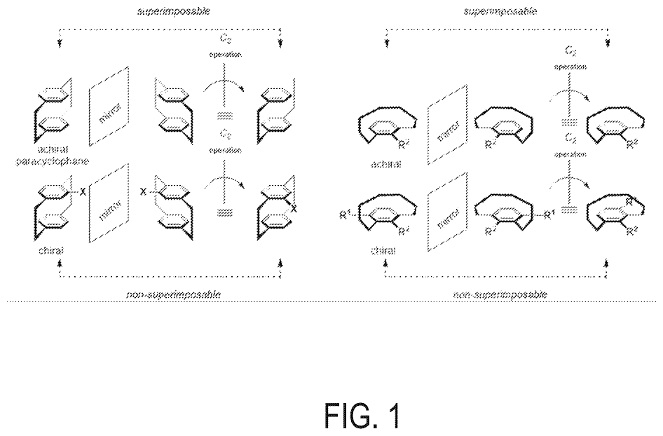

The present disclosure also involves macrocyclization. Macrocyclizations are difficult to predict as many techniques that work for small ring closures, or that work for intermolecular reactions, do not work in the context of macrocyclization. The presently described synthetic method surprising obtains high yields of macrocyclized cyclophanes via a chiral ligand mediated palladium-catalyzed coupling. Yet more surprisingly, the present approach can proceed in enantiomerically selective manner. Planar chiral macrocyclophanes represent a type of chiral molecule where chirality is exhibited due to a non-superimposable planar configuration. See, FIG. 1. This type of chirality is difficult to impart onto compounds in a controlled manner.

The present disclosure provides a method that involves contacting a substrate with a palladium cross-coupling catalyst, base, and a chiral ligand, wherein the substrate has a structure according to Formula I.

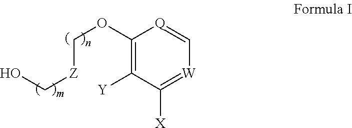

In various aspects of formula I, Q is CH or N and W is CH or N. In some aspects, at least one of Q and W is N. In some aspects, at least one of Q and W is CH. In some aspects, Q is N and W is CH. In other examples, Q is CH and W is N.

In various aspects, X is a halide. For example, the halide can be chloride, bromide, iodide or fluoride. In some aspects, the halide is bromide or iodide. In some examples, the halide is chloride. In yet further examples, the halide can be a halide equivalent, such as mesylate, triflate, or the like.

In various aspects of formula I, Y is a sterically bulky group. Examples of a sterically bulky group include tert-butyl, isopropyl, isobutyl, sec-butyl, neopentyl, adamantyl, norbornyl, cyclopentyl, cyclohexyl, phenyl, naphthyl, benzyl, ethylphenyl, nitrophenyl, methoxyphenyl, fluorophenyl, chlorophenyl, bromophenyl, iodophenyl, trifluoromethylphenyl, difluoromethylphenyl, trityl, and acyl, ester, and amide derivatives of the same. Further examples of a sterically bulky group include sterically bulky acyl, ester, and amide groups including groups where Y is —C(O)R1, —C(O)NH—R1, —C(O)N(R1)₂, —C(O)O—R1, —NHC(O)—R1, —OC(O)—R1, —OC(O)O—R1, and —NHC(O)NH—R1. R1 can be C2-C10 alkyl, including linear or branched alkyl. R1 can be n-ethyl, n-propyl, n-burtyl, tert-butyl, isopropyl, isobutyl, sec-butyl, norpentyl, adamantyl, norbornyl, cyclopentyl, cyclohexyl, phenyl, naphthyl, benzyl, ethylphenyl, nitrophenyl, methoxyphenyl, fluorophenyl, chlorophenyl, bromophenyl, iodophenyl, trifluoromethylphenyl, difluoromethylphenyl, or trityl.

In various aspects of Formula I, Y comprises a branched C3-C10 alkyl, cycloalkyl, aryl, heteroaryl, or non-aromatic heterocyclyl. In various aspects of Formula I, Y is a branched C3-C10 alkyl, cycloalkyl, aryl, heteroaryl, or non-aromatic heterocyclyl.

In various aspects of Formula I, Y has the structure:

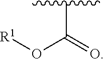

In various aspects of Formula I, Y has the structure:

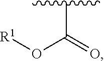

wherein R1 is a branched C3-C6 alkyl group.

In various aspects of formula I, m is 0 to 10 and n is 0 to 10. In various aspects, m is 0, 1, or 2 and n is 6, 7, 8, 9, or 10. In various aspects, n is 0, 1, or 2 and m is 6, 7, 8, 9, or 10. In various aspects of formula I, m is 3 to 7 and n is 3 to 7. For example, m can be 3, 4, 5, 6, or 7, and n can be 3, 4, 5, 6, or 7. The m and n groups are independently defined and can be the same or they can be different. For example, m can be 3 and n can be 3; m can be 3 and n can be 4; m can be 3 and n can be 5; m can be 3 and n can be 6; m can be 3 and n can be 7; m can be 4 and n can be 3; m can be 4 and n can be 4; m can be 4 and n can be 5; m can be 4 and n can be 6; m can be 4 and n can be 7.

In various aspects of formula I, Z is a divalent linking group. The divalent linking group is a group with the ability to form at least two bonds so as to link the (CH2)n and (CH2)m moieties.

In various aspects of Formula I, the divalent linking group can be an optionally substituted divalent alkyl, alkenyl, or alkynyl group, for example, a linear alkylene, branched alkylene, cycloalkylene, aromatic, heteroaromatic, alkenylene, alkynylene, aralkylene, or a heterocyclic group. Examples include —CH2-, —C(CH3)2-, —C(C2H5)2-, —C6H4-, —CH═CH—, —C≡C—, —C(CH3)═CH—, —C(C2H5)═CH—, —CH2CH2-, —CH2CH(CH3)-, —CH2C(CH3)2-, —CH2C(C2H5)2-, —CH2C6H4-, —CH2CH═CH—, —CH2C≡C—, —CH2C(CH3)═CH—, —CH2C (C2H5)═CH—, and others.

In various aspects of Formula I, the divalent linking group can be an aromatic or heteroatomic group. Examples of aromatic groups include phenylene, naphthylene, anthracene, and pyridine. Heteroaromatic groups can include pyrrole, imidazole, furan, thiophene, pyrazole, oxazole, thiazole, isothiazole, isoxazole, pyridine, pyridazine, pyrimidine, pyrazine, triazine, quinoline, isoquinoline, indole, benzofuran, and benzothiophene.

In various aspects of Formula I, the divalent linking group can be a heteroatom, such as —O—, —S—, or NH.

In various aspects of Formula I, the divalent linking group contains at least one aryl or heteroaromatic group. In various other aspects, the divalent linking group is free of an aryl or heteroaromatic group. In various further aspects, the divalent linking group is a linear chain, such as a linear hydrocarbon chain. In various further aspects, the divalent linking group consists of an aromatic moiety. aromatic is a linear chain, such as a linear hydrocarbon chain.

In various aspects of Formula I, the divalent linking group is optionally substituted, for example with one or more halogen atoms, nitro groups, cyano groups, alkyl, or alkenyl groups, or other substituents. In various aspects, the divalent linking group Z can contain two, three, four, or more of the aforementioned divalent moieties such that they together serve as the divalent group linking the (CH2)n and (CH2)m moieties. For example, Z can be a —C6H4-CH2-C6H4- group.

In various aspects of Formula I, the divalent linking group contains at least or about 3, 4, 5, 6, 7, 8, 9, or 10 carbon atoms. In various aspects of Formula I, the divalent linking group contains a linear backbone between the points of attachment linking the (CH2)n and (CH2)m moieties. The linear backbone can contain at least or about 1, 2, 3, 4, 5, 6, 7, 8, 9, or 10 atoms. The linear backbone can contain at least or about 1, 2, 3, 4, 5, 6, 7, 8, 9, or 10 carbon atoms.

In various aspects of Formula I, Z is an optionally substituted phenylene linker. In various aspects of Formula I, Z is an optionally substituted divalent alkyl group. In various aspects of Formula I, Z is an optionally substituted C3-C8 alkyl linker. In various aspects of Formula I, Z provides a linear backbone of at least three atoms between points of attachment. In various aspects of Formula I, wherein n, m, and Z together provide a linear backbone of at least 8 atoms between points of attachment. In various aspects of Formula I, Z comprises an aromatic group.

In various aspects of Formula I, a linear chain of atoms between (but not including) X and the terminal OH moiety of Formula I provides a 10, 11, 12, 13, 14, 15, 16, 17, 18, 19, or 20

The various methods described herein utilize a palladium cross-coupling catalyst. In various aspects, the palladium cross-coupling catalyst is [(Allyl)PdCl]₂ or other palladium source. Palladium sources can also include other [(alkenyl)PdX]₂-type catalysts, for example, [(n-Butenyl)PdCl]₂, [(pi-cinnamyl)PdCl]₂, Pd₂(dba)₃, bis(benzonitrile)PdCl₂, Pd(OAc)₂, and Pd(PPh₃)₄). Other examples include Pd(PPh₃)₂X₂, Pd(dppf)₂X₂, Pd(PCy₃)₂X₂, Pd(PR₃)₂X₂, wherein R is an alkyl or aromatic group, X is a halogen, acetate, or the like.

In various aspects, the palladium cross-coupling catalyst is utilized at approximately 0.01 mol %, to approximately 50 mol % relative to the starting material substrate. In various aspects, the palladium cross-coupling catalyst is utilized at 0.01 mol %, 0.02 mol %, 0.03 mol %, 0.04 mol %, 0.05 mol %, 0.06 mol %, 0.07 mol %, 0.08 mol %, 0.09 mol %, 0.1 mol %, 0.2 mol %, 0.3 mol %, 0.4 mol %, 0.5 mol %, 0.6 mol %, 0.7 mol %, 0.8 mol %, 0.9 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, 15 mol %, 16 mol %, 17 mol %, 18 mol %, 19 mol %, 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, or 50 mol % relative to the starting material substrate. For example, the palladium cross-coupling catalyst can be utilized at approximately 10 mol % relative to the starting material substrate. The chiral ligand can be utilized at approximately 1:2 Pd/ligand ratio to 1:10 Pd/Ligand Ratio. For example, the Pd/ligand ratio can be 1:2, 1:3, 1:4, 1:5, 1:6, 1:7, 1:8, 1:9, or 1:10.

In various aspects, the palladium cross-coupling catalyst is a diphosphine ligand. In various aspects, the palladium cross-coupling catalyst is an aromatic moiety. In various aspects, the palladium cross-coupling catalyst is a ferrocene-containing ligand. In various aspects, the palladium cross-coupling catalyst is a JosiPhos-type ligand.

Various bases can be used in the presently described method. The base can be a mild base suitable for palladium cross-coupling and palladium cross-coupling catalysts. For example, the base can be Cs₂CO₃, NaO′Bu, CsF, or KOAc. In various aspects, the base is an inorganic carbonate. In various aspects, the base is a cesium salt. In various aspects, the base is an inorganic compound. The base can be utilized at any suitable concentration. The base can be utilized at about 2, 3, 4, or 5 equivalents relative to the substrate.

In various aspects, the palladium cross-coupling catalyst, base, chiral ligand, and substrate can be heated to an elevated temperature of about 50° C. or higher. For example, the elevated temperature can be about or at least 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C. In other aspects, the palladium cross-coupling catalyst, base, chiral ligand, and substrate can be maintained at or near room temperature, mild cooling, or about or at least 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., or 29° C.

In various aspects, the palladium cross-coupling catalyst, base, chiral ligand, and substrate can be stirred when contacted together. For example, the palladium cross-coupling catalyst, base, chiral ligand, and substrate are stirred with a magnetic stirrer or mechanical stirrer. The palladium cross-coupling catalyst, base, chiral ligand, and substrate can be stirred together at an RPM of at least 100. In various aspects, the RPM can be about or at least 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 5000, or a range of any of the preceding values.

The palladium cross-coupling catalyst, base, chiral ligand, and substrate can be contacted together all at once, or sequentially in any combination or permutation of orders.

In various aspects, cyclizing the compound of Formula I in the presently described method results in a macrocyclic cyclophane having a ring size of 10, 11, 12, 13, 14, 15, 16, 17, 18, 19, or 20 atoms.

The disclosure provides a compound having a structure according to Formula II, or a salt thereof:

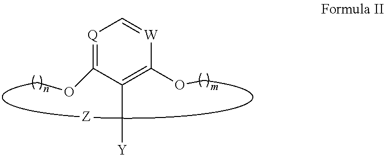

The disclosure also provides a compound having a structure according to Formula III, or a salt thereof:

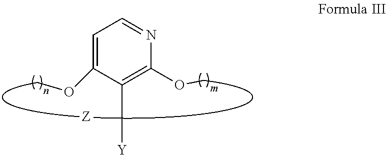

In various aspects of the compound of Formula II and Formula III, Q is CH or N and W is CH or N. In some aspects, at least one of Q and W is N. In some aspects, at least one of Q and W is CH. In some aspects, Q is N and W is CH. In other examples, Q is CH and W is N.

In various aspects of the compound of Formula II and Formula III, Y is a sterically bulky group. Examples of a sterically bulky group include tert-butyl, isopropyl, isobutyl, sec-butyl, neopentyl, adamantyl, norbornyl, cyclopentyl, cyclohexyl, phenyl, naphthyl, benzyl, ethylphenyl, nitrophenyl, methoxyphenyl, fluorophenyl, chlorophenyl, bromophenyl, iodophenyl, trifluoromethylphenyl, difluoromethylphenyl, trityl, and acyl, ester, and amide derivatives of the same. Further examples of a sterically bulky group include sterically bulky acyl, ester, and amide groups including groups where Y is —C(O)R1, —C(O)NH—R1, —C(O)N(R1)₂, —C(O)O—R1, —NHC(O)—R1, OC(O)—R1, —OC(O)O—R1, and —NHC(O)NH—R1. R1 can be C2-C10 alkyl, including linear or branched alkyl. R1 can be n-ethyl, n-propyl, n-burtyl, tert-butyl, isopropyl, isobutyl, sec-butyl, norpentyl, adamantyl, norbornyl, cyclopentyl, cyclohexyl, phenyl, naphthyl, benzyl, ethylphenyl, nitrophenyl, methoxyphenyl, fluorophenyl, chlorophenyl, bromophenyl, iodophenyl, trifluoromethylphenyl, difluoromethylphenyl, or trityl.

In various aspects of the compound of Formula II and Formula III, Y comprises a branched C3-C10 alkyl, cycloalkyl, aryl, heteroaryl, or non-aromatic heterocyclyl. In various aspects of Formula I, Y is a branched C3-C10 alkyl, cycloalkyl, aryl, heteroaryl, or non-aromatic heterocyclyl.

In various aspects of the compound of Formula II and Formula III, Y has the structure:

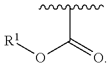

In various aspects of the compound of Formula II and Formula III, Y has the structure:

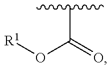

wherein R1 is a branched C3-C6 alkyl group.

In various aspects of the compound of Formula II and Formula III, m is 0 to 10 and n is 0 to 10. In various aspects, m is 0, 1, or 2 and n is 6, 7, 8, 9, or 10. In various aspects, n is 0, 1, or 2 and m is 6, 7, 8, 9, or 10. In various aspects of formula I, m is 3 to 7 and n is 3 to 7. For example, m can be 3, 4, 5, 6, or 7, and n can be 3, 4, 5, 6, or 7. The m and n groups are independently defined and can be the same or they can be different. For example, m can be 3 and n can be 3; m can be 3 and n can be 4; m can be 3 and n can be 5; m can be 3 and n can be 6; m can be 3 and n can be 7; m can be 4 and n can be 3; m can be 4 and n can be 4; m can be 4 and n can be 5; m can be 4 and n can be 6; m can be 4 and n can be 7.

In various aspects of the compound of Formula II and Formula III, Z is a divalent linking group. The divalent linking group is a group with the ability to form at least two bonds so as to link the (CH2)n and (CH2)m moieties.

In various aspects of the compound of Formula II and Formula III, the divalent linking group can be an optionally substituted divalent alkyl, alkenyl, or alkynyl group, for example, a linear alkylene, branched alkylene, cycloalkylene, aromatic, heteroaromatic, alkenylene, alkynylene, aralkylene, or a heterocyclic group. Examples include —CH2—, —C(CH3)2-, —C(C2H5)2-, —C6H4-, —CH═CH—, —C≡C—, —C(CH3)═CH—, —C(C2H5)═CH—, —CH2CH2-, —CH2CH(CH3)-, —CH2C(CH3)2-, —CH2C(C2H5)2-, —CH2C6H4-, —CH2CH═CH—, —CH2C≡C—, —CH2C(CH3)═CH—, —CH2C(C2H5)═CH—, and others.

In various aspects of the compound of Formula II and Formula III, the divalent linking group can be an aromatic or heteroatomic group. Examples of aromatic groups include phenylene, naphthylene, anthracene, and pyridine. Heteroaromatic groups can include pyrrole, imidazole, furan, thiophene, pyrazole, oxazole, thiazole, isothiazole, isoxazole, pyridine, pyridazine, pyrimidine, pyrazine, triazine, quinoline, isoquinoline, indole, benzofuran, and benzothiophene.

In various aspects of the compound of Formula II and Formula III, the divalent linking group can be a heteroatom, such as —O—, —S—, or NH.

In various aspects of the compound of Formula II and Formula III, the divalent linking group contains at least one aryl or heteroaromatic group. In various other aspects, the divalent linking group is free of an aryl or heteroaromatic group. In various further aspects, the divalent linking group is a linear chain, such as a linear hydrocarbon chain. In various further aspects, the divalent linking group consists of an aromatic moiety. aromatic is a linear chain, such as a linear hydrocarbon chain.

In various aspects of the compound of Formula II and Formula III, the divalent linking group is optionally substituted, for example with one or more halogen atoms, nitro groups, cyano groups, alkyl, or alkenyl groups, or other substituents. In various aspects, the divalent linking group Z can contain two, three, four, or more of the aforementioned divalent moieties such that they together serve as the divalent group linking the (CH2)n and (CH2)m moieties. For example, Z can be a -C6H4-CH2-C6H4-group.

In various aspects of the compound of Formula II and Formula III, the divalent linking group contains at least or about 3, 4, 5, 6, 7, 8, 9, or 10 carbon atoms. In various aspects of Formula I, the divalent linking group contains a linear backbone between the points of attachment linking the (CH2)n and (CH2)m moieties. The linear backbone can contain at least or about 1, 2, 3, 4, 5, 6, 7, 8, 9, or 10 atoms. The linear backbone can contain at least or about 1, 2, 3, 4, 5, 6, 7, 8, 9, or 10 carbon atoms.

In various aspects, the compound of Formula II or Formula III represent a macrocyclic cyclophane having a ring size of 10, 11, 12, 13, 14, 15, 16, 17, 18, 19, or 20 atoms. In certain aspects, the macrocyclic cyclophane has a ring size of at least 12, 13, 14, or 15, or less than 17, 18, or 19.

In various aspects of the compound of Formula II and Formula III, Z is an optionally substituted phenylene linker. In various aspects of Formula I, Z is an optionally substituted divalent alkyl group. In various aspects of Formula I, Z is an optionally substituted C3-C8 alkyl linker. In various aspects of Formula I, Z provides a linear backbone of at least three atoms between points of attachment. In various aspects of Formula I, wherein n, m, and Z together provide a linear backbone of at least 8 atoms between points of attachment. In various aspects of Formula I, Z comprises an aromatic group.

In various aspects, the compound of Formula II or Formula III has the structure:

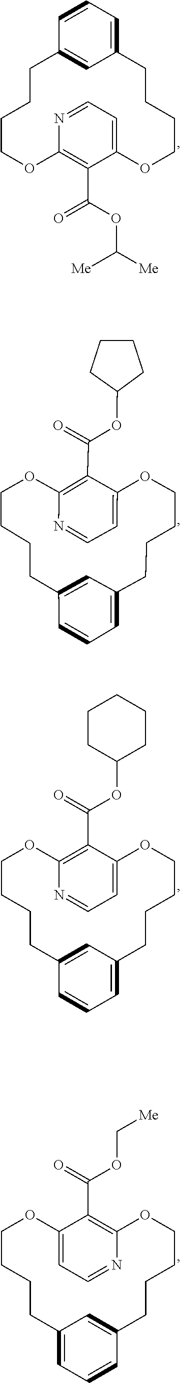 [SMILES: CC(C)OC(=O)c1c2ccnc1OCCCCc1cccc(c1)CCCCO2 ; O=C(OC1CCCC1)c1c2ccnc1OCCCCc1cccc(c1)CCCCO2 ; O=C(OC1CCCCC1)c1c2ccnc1OCCCCc1cccc(c1)CCCCO2 ; CCOC(=O)c1c2ccnc1OCCCCc1cccc(c1)CCCCO2]

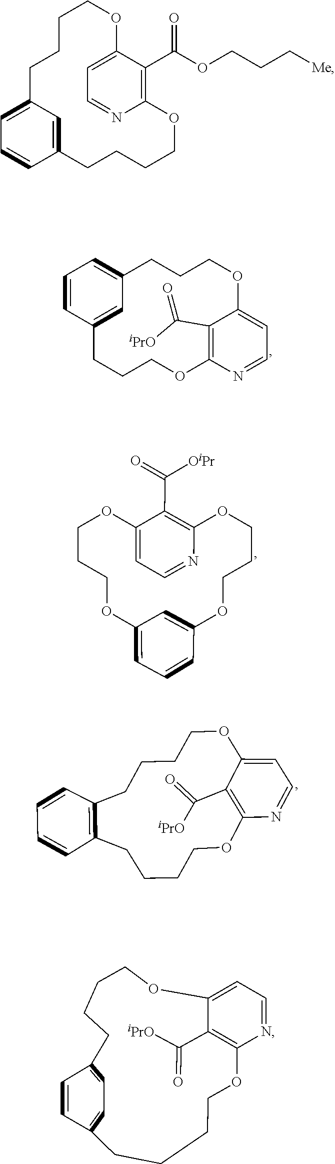 [SMILES: CCCCOC(=O)c1c2ccnc1OCCCCc1cccc(c1)CCCCO2 ; CC(C)OC(=O)c1c2ccnc1OCCCc1cccc(c1)CCCO2 ; CC(C)OC(=O)c1c2ccnc1OCCCOc1cccc(c1)OCCCO2 ; CC(C)OC(=O)c1c2ccnc1OCCCCc1ccccc1CCCCO2 ; CC(C)OC(=O)c1c2ccnc1OCCCCc1ccc(cc1)CCCCO2]

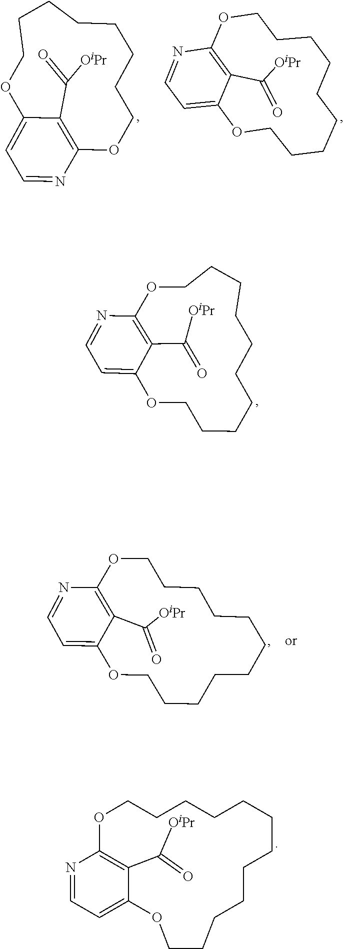

In various aspects, the compound of Formula II or Formula III is a compound having the structure:

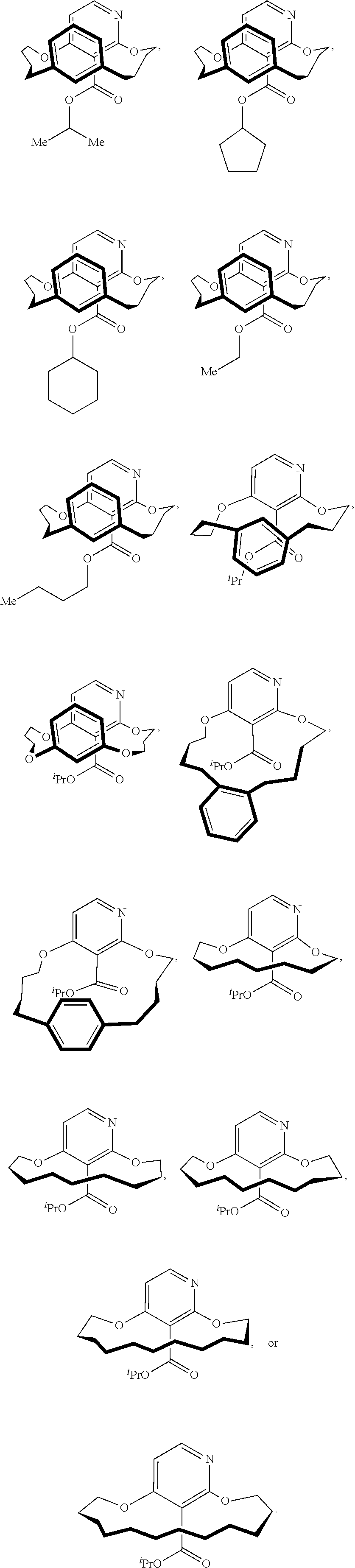

In various aspects, the compound of Formula II or Formula III is a racemate, a non-racemic mixture of stereoisomers, an (Rp)-enantiomer, or an (Sp)-enantiomer. For example, the compound can be an (Sp)-enantiomer. In other examples, the compound can be an (Rp)-enantiomer.

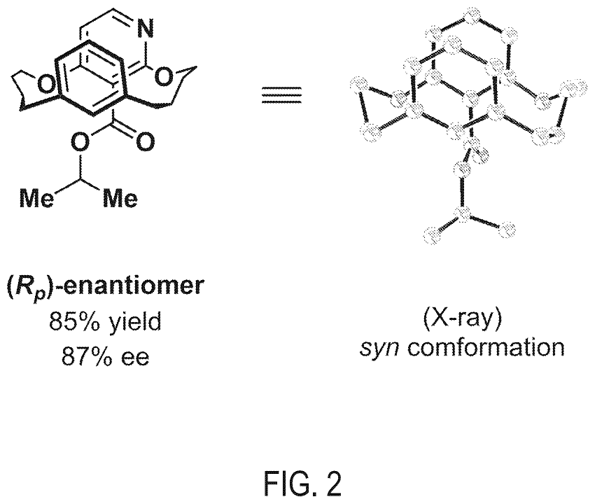

See, for example, FIG. 2, which demonstrates the configuration that such cyclophanes can exhibit.

In various aspects, the compound of Formula II or Formula III is in a syn-conformation.

In various aspects, the compound of Formula II or Formula III is in an anti-conformation.

### EXAMPLES

Various embodiments of the present invention can be better understood by reference to the following Examples which are offered by way of illustration. The present invention is not limited to the Examples given herein.

### A. Materials and Methods

Room temperature is defined as 23° C. All reagents were purchased from commercial suppliers and used without further purification, unless otherwise noted. Tetrahydrofuran and toluene were obtained from an 800 L Solvent Purification System by Pure Process Technology, in which the solvent was dispensed under an atmosphere of Ar. Where indicated, experiments were carried out in a nitrogen-filled mBraun glovebox. All other solvents were purchased from commercial suppliers and used without further purification, unless otherwise noted. All the yields were isolated yields. Rotary evaporation was carried out at 40° C.

Routine 1H NMR spectra was recorded on Bruker 400 or 600 MHz spectrometers at ambient temperature unless otherwise stated. All NMR solvents were purchased from Cambridge Isotope Laboratories and used without further purification. Chloroform-d and DMSO-d6 were stored at ambient temperature. Spectra were processed using MestReNova 14.0.1 using the automatic phasing and polynomial baseline correction capabilities. Splitting was determined using the automatic multiplet analysis function with manual intervention as necessary. Spectral data are reported as follows: chemical shift (multiplicity[singlet(s), broad singlet (br s), doublet (d), triplet (t), quartet (q), pentet (p), septet (sept), multiplet (m), doublet of doublets (dd), doublet of doublet of doublets (ddd), doublet of triplet of doublets (dtd), doublet of doublet of doublet of doublets (dddd), doublet of triplets (dt), triplet of doublets (td), etc.], coupling constant, integration). Chemical shifts are reported in ppm (δ), and coupling constants are reported in Hz. 1H Resonances are referenced to solvent residual peaks for CDCl3 (7.26 ppm), acetone (2.05 ppm), and DMSO (2.50 ppm). 13C Resonances are referenced to solvent residual peaks for CDCl3 (77.16 ppm). Note: Small deviations in chemical shifts may be observed depending on the concentration of NMR samples.

Analytical thin-layer chromatography was performed using 60 Å Silica Gel F254 pre-coated plates (0.25 mm thickness). TLC plates were visualized by irradiation with a UV lamp. Normal-phase column chromatography was performed using 60 Å Silica Gel (32-62 micron) with an appropriate mobile phase composition and gradient. Automated column chromatography was performed using a CombiFlash NextGen 300+ System by Teledyne ISCO on RediSep Rf Gold silica gel columns or RediSep Rf disposable flash columns. Mass spectrometry was performed using the Agilent 6540 UHD Accurate-Mass Q-TOF LC/MS. Infrared spectra were recorded on a Bruker Tensor 37 ATR/FT-IR spectrometer, and vmax are reported in cm−1. Enantiomeric excesses were determined by HPLC using Daicel chiral stationary phase columns by comparing the samples with the corresponding racemic samples at 25° C. Elution details specified in each entry. Optical rotation data were recorded on JASCO Model DIP 370. Configuration was determined by X-ray structures. All chiral ligands were purchased from Stem Chemicals and Combi-Blocks and used without further purification. All Pd sources were purchased from Sigma-Aldrich unless otherwise noted.

### B. Precursor Synthesis

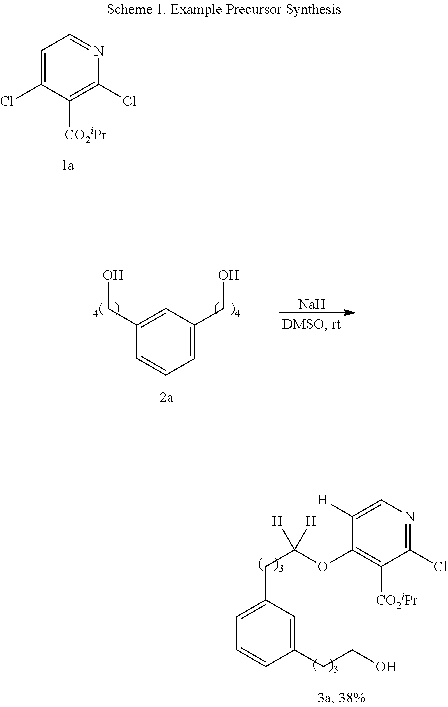

Modular syntheses of a linear precursor for enantioselective macrocyclization were prepared from a 2,3,4-trisubstituted dichloropyridine 1a and a bridging chain 2a, which were assembled with a C4-selective SNAr reaction. The regioselectivity of the SNAr reaction was verified by NOESY NMR, which showed cross peaks between the methylene group on the bridging chain and the H at the 5-position of the pyridine. This synthesis strategy proved to be generally applicable to all substrates examined.

### C. Macrocycle Synthesis

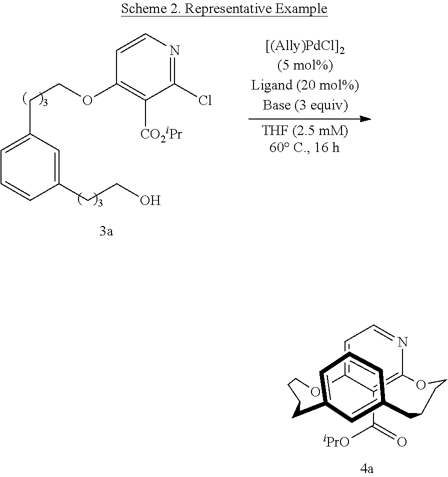

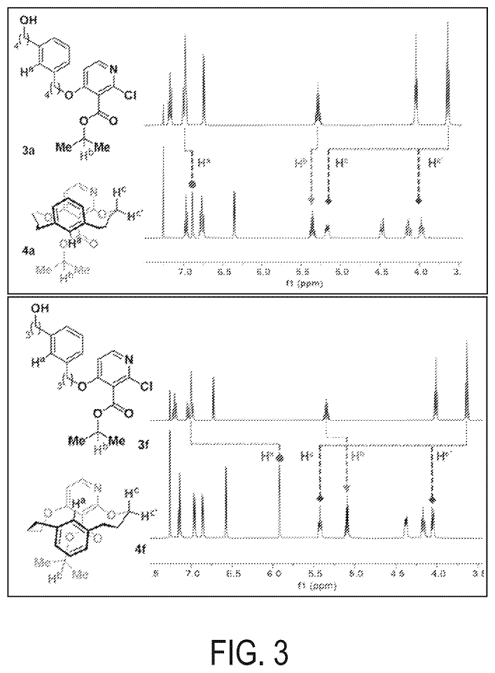

Initial attempts at enantioselective macrocyclization using Pd, a JosiPhos-type ligand (“L1”), and NaOtBu resulted in 18% yield of the desired product with low enantioselectivity. A control experiment without Pd and a ligand revealed that a racemic SNAr reaction was competitive when NaO′Bu was used but not with the less basic Cs₂CO₃. The reaction using [Pd(allyl)Cl]₂ and Cs₂CO₃ gave the desired product 4a in 85% yield and 93% ee at a concentration of 2.5 mM. The orientation of the resulting product was configured by NMR. See, FIG. 3.

Other JosiPhos-type ligands were tested (entries 3-4), chiral ligand scaffolds (entries 5-6), and bases (entries 7-8) were not as effective as those of the JosiPhos-type ligands. Running the reaction at lower or higher temperatures gave lower yields and/or ee. Larger scale (0.1 mmol) saw a decrease in yield as we increased the concentration from 2.5 to 20 mM. A high stir rate was advantageous for reproducible yields and enantioselectivities A 1:2 Pd/ligand ratio was also advantageous for reproducibility. Lower Pd/ligand ratios led to visible formation of Pd black.

TABLE 1
Test Ligands

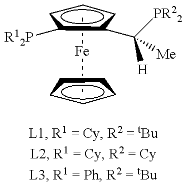

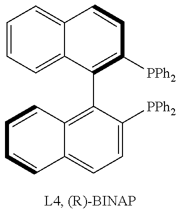

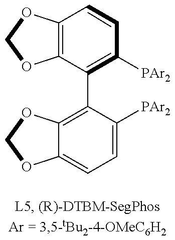

| Entry | Ligand | Base | Yield [symbol] | ee | Configuration |
|---|---|---|---|---|---|
| 1 | L1 | NaO^{t}Bu | 18% | 13% | Sₚ |
| 2 | L1 | Cs₂CO₃ | 85% | 93% | Rₚ |
| 3 | L2 | Cs₂CO₃ | 19% | 40% | Rₚ |
| 4 | L3 | Cs₂CO₃ | 37% | 56% | Sₚ |
| 5 | L4 | Cs₂CO₃ | 62% | 26% | Rₚ |
| 6 | L5 | Cs₂CO₃ | 38% | 34% | Sₚ |
| 7 | L1 | CsF | 15% | 75% | Rₚ |
| 8 | L1 | KOAc | 52% | 22% | Rₚ |

[symbol]

indicates data missing or illegible when filed

### D. Reaction Development

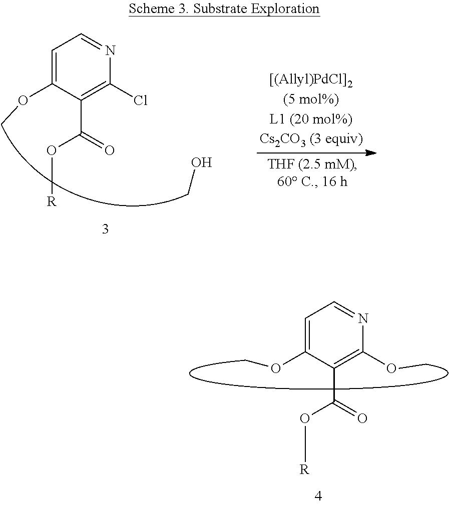

The reaction scope was then explored by varying the ester group, ring size, and type of bridging chain. The enantioselective macrocyclization can generate[5.5]meta-cyclophanes 4a-4e with different ester groups in 65-85% yields and in 87-92% ee. Other macrocyclic metacyclophanes can also be synthesized, including[4.4]metacyclophane 4f (77% yield, 84% ee), a cyclophane 4g with four arylalkylether linkages (59% yield, 79% ee), a[5.5]metaorthocyclophane 4h (76% yield, 79% ee), and a [5.5]metaparacyclophane 4i (61% yield, 83% ee). The enantioselective macrocyclizations still proceed in good yields when more conformationally flexible, all-alkyl bridging chains are used, generating 13- to 17-membered macrocycles 4j-4n in 64-77% yields and 83-90% ee. Smaller 11- and 12-membered macrocycles predominantly included dimeric products. Accordingly, the method provided 16-membered macrocycles with different esters, 14- to 17-membered macrocycles with an aromatic (e.g., benzene) linker, and 13- to 17-membered macrocycles with a linear straight chain (e.g., alkyl) linker. All reactions were run on a 0.100 mmol scale in THF (2.5 mM). Isolated yields were obtained as absolute configurations of 4a and 4f were assigned by X-ray crystallography; the absolution configuration of the other products was assigned by analogy. Without intending to limit by theory, it is thought that cyclization occurred via a 4-to-2 cyclization via the substituent on the C4-position to the reacting C2-position. Additionally, racemization tests on 4a and 4n showed that the macrocycles underwent decomposition before racemization when heated in toluene at 100° C. for 20 h. The crystal structures also showed that [4.4]metacyclophane 4f and [5.5]metacyclophane 4a adopt anti-and synconformations, respectively, in the solid state.

### E. Double C—O Cross-Couplings

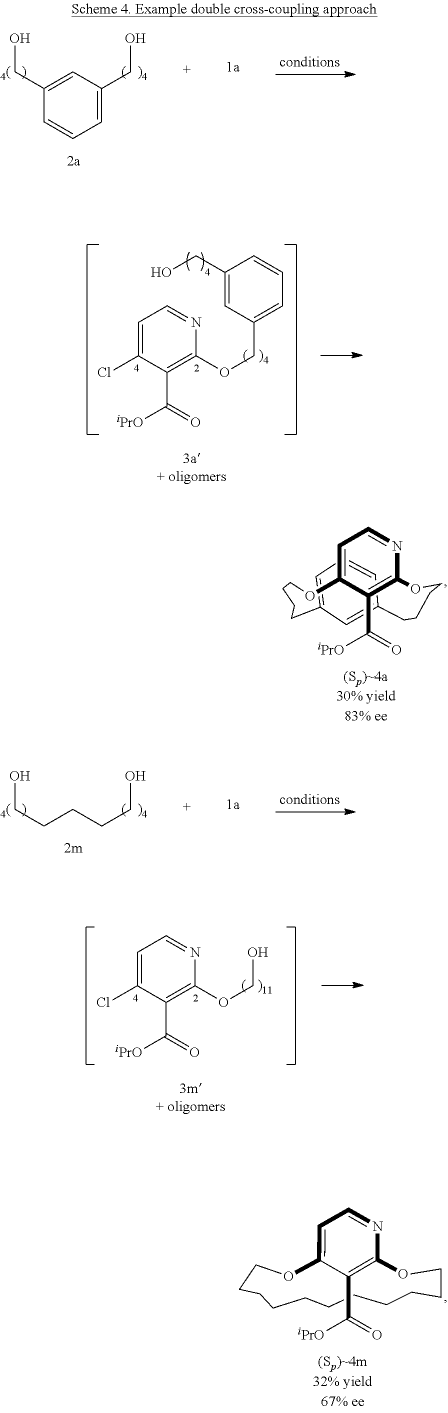

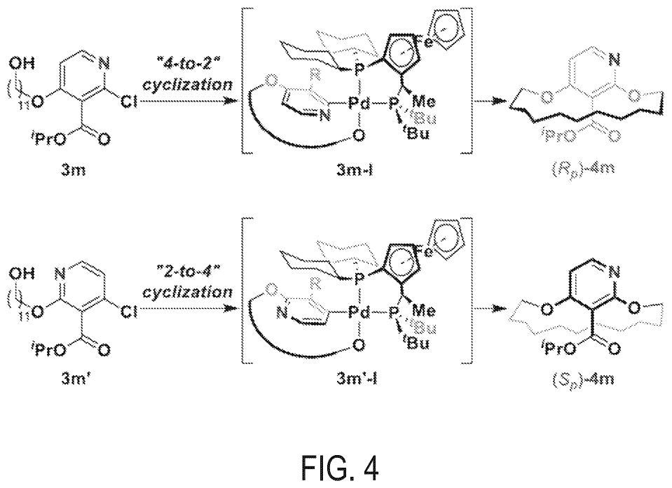

Having successfully developed access the planar chiral metacyclophanes, we next attempted to streamline the synthesis using a double C-O cross-coupling reaction between 1a and 2a and between 1a and 2m. Consistent with literature reports, C-O cross-coupling occurred selectively at the C2-position of the pyridine to generate 3a′ and 3m′, respectively, within 2 h. This was followed by enantioselective macrocyclization onto the C4-position of the pyridyl group, which gave (Sp)-4a (30% yield, 83% ee) and (Sp)-4m (32% yield, 67% ee), respectively. We isolated 3m′ from the reaction between 1a and 2m in low yield, indicating that competitive formation of oligomers was occurring in the initial intermolecular coupling, which required a higher concentration of 20 mM. This was further verified by isolating 3a′ and 3m′ and subjecting them to the double cross-coupling conditions to give (Sp)-4a (70% yield, 85% ee) and (Sp)-4m (61% yield, 63% ee), respectively. These yields are comparable with the reactions starting from 3a and 3m to give (Rp)-4a (72% yield, 85% ee) and (Rp)-4m (69% yield, 74% ee) under the same conditions, which in each case were [(Allyl)PdCl]2 (5 mol %), L1 (20 mol %), Cs2CO3 (3 equiv), THF (20 mM), 60° C., 16 h. We also made two observations on the differences in the rate of cyclizations: (i) the “2-to-4” cyclizations of 3a′ and 3m′ required longer times to reach full conversion than “4-to-2” cyclizations of 3a and 3m, as would be expected based on the higher reactivity of the C2 chloride in Pd-catalyzed cross-coupling reactions; 25 (ii) the conformationally flexible all-alkyl bridging chains in 3m and 3m′ lead to a slower macrocyclization. We observed that the sense of enantioinduction was reversed when the cyclization was changed from a “4-to-2” cyclization to a “2-to-4” cyclization using the same enantiomer of L1. Without intending to limit by theory, it appears that the position of the nitrogen atom affects the orientation of the oxidative addition complex. See, FIG. 4. In these complexes, the aryl group is trans to the bulky PtBu2 moiety, and the top half of the pyridine plane is in a sterically crowded environment imposed by the ligand structure. The chiral Pd complex induces enantioselectivity by favoring cyclization of the bridging chain from the bottom face of the prochiral pyridyl plane to avoid steric clash with the cyclohexyl groups on L1, thus generating 3m-I and 3m′-I, which produce opposite enantiomers of the cyclophane product after reductive elimination.

TABLE 2
Test Macrocycles

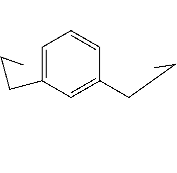 [SMILES: CC(C)OC(=O)c1c2ccnc1OCCc1cccc(c1)CCO2]

(Rₚ)-4a
65% yield
87% ee

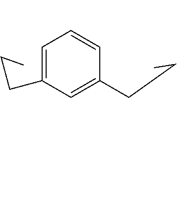 [SMILES: O=C(OC1CCCC1)c1c2ccnc1OCCc1cccc(c1)CCO2]

(Rₚ)-4b
79% yield [symbol]
2% ee

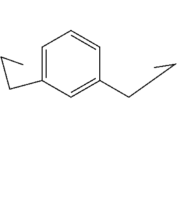 [SMILES: O=C(OC1CCCCC1)c1c2ccnc1OCCc1cccc(c1)CCO2]

(Rₚ)-4c
70% yield [symbol]
8% ee

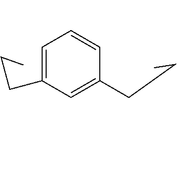 [SMILES: CCOC(=O)c1c2ccnc1OCCc1cccc(c1)CCO2]

(Rₚ)-4d
65% yield
89% ee

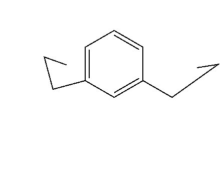 [SMILES: CCCCOC(=O)c1c2ccnc1OCCc1cccc(c1)CCO2]

(Rₚ)-4e
69% yield
87% ee

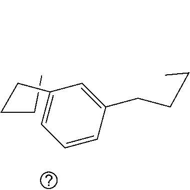 [SMILES: COC(=O)c1c2ccnc1OCCCc1cccc(c1)CCCO2]

(R)ₚ)-[4.4]metametacyclophane 4f
77% yield
84% ee

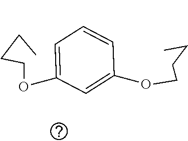 [SMILES: CC(=O)c1c2ccnc1OCCCOc1cccc(c1)OCCCO2]

(Rₚ)-[5,5]metametacyclophane 4g
59% yield
7 [symbol] % ee

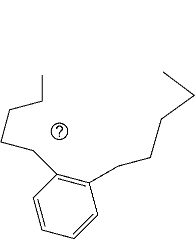 [SMILES: CC(=O)c1c2ccnc1OCCCCc1ccccc1CCCCO2]

(Rₚ)-[5.5]meta [symbol] rthocyclophane 4h
76% yield
79% ee

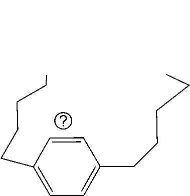 [SMILES: CC(=O)c1c2ccnc1OCCCCc1ccc(cc1)CCCCO2]

(Rₚ)-[5.5]metaparacyclophane 4i
81% yield
83% ee

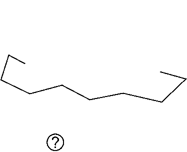 [SMILES: CC(=O)c1c2ccnc1OCCCCCCCCO2]

13-membered ring (Rₚ)-4j
64% yield
83% ee

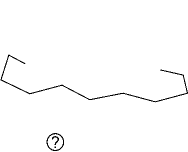 [SMILES: CC(=O)c1c2ccnc1OCCCCCCCCCO2]

14-membered ring (Rₚ)-4k
69% yield
90% ee

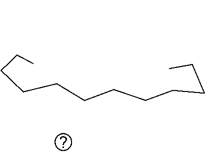 [SMILES: CC(=O)c1c2ccnc1OCCCCCCCCCCO2]

15-membered ring (Rₚ)-4l
71% yield
88% ee

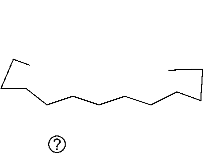 [SMILES: CC(=O)c1c2ccnc1OCCCCCCCCCCCO2]

16-membered ring (Rₚ)-4m
77% yield
87% ee

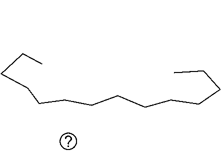 [SMILES: CC(=O)c1c2ccnc1OCCCCCCCCCCCCO2]

17-membered ring (Rₚ)-4n [symbol]
9% yield
85% ee [symbol]
indicates data missing or illegible when filed

### F. Ligand Screen

A 20-mL vial was charged with a magnetic stir bar, and 3a (0.011 g, 0.025 mmol). The vial was brought into a glovebox, then Cs2CO3 (0.025 g, 0.075 mmol, 3.0 equiv), 1.00 mL of a THF solution of [(allyl)PdCl]2 (1.25 mM, 1.25 μmol, 5.0 mol %), a ligand (0.0050 mmol, 20.0 mol %), and THF (9.0 mL) were added. The vial was sealed and brought outside and placed in an aluminum heating block preheated at 60° C. The mixture was stirred (800 rpm) for 16 h. After cooling to room temperature, the mixture was filtered through a glass pipet packed with Celite (1×2 cm) using 5.0 mL of THF for washing. The filtrate was concentrated by rotary evaporator, and the residues were loaded onto a RediSep Gold® Normal-Phase Silica 4-gram column. The column was eluted using a Combiflash with a mixture of 30% EtOAc/hexanes for 3.0 CV (13 mL/minute). The product started to elute after 2.0 CV. The combined product-containing fractions were collected. The solvent was removed using rotary evaporator and the residue was further dried under high vacuum to give 4a as a colorless oil. Note: Cs2CO3 was ground with a pestle and mortar in the glovebox to a fine powder. It is advantageous to apply a stir rate of 800 rpm to the reactions to ensure reproducibility.

Scheme 5
Ligand Screening Conditions

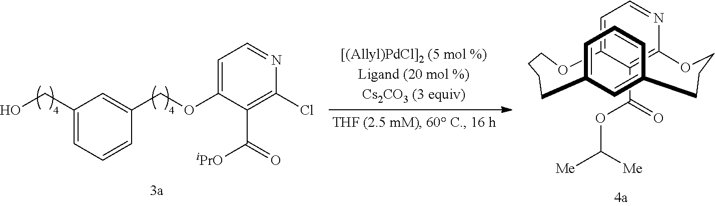

| Entry | Ligand | Yield (%) | ee (%) | Configuration |
|---|---|---|---|---|
| 1 | L1 | 85 | 93 | Rₚ |
| 2 | L2 | 19 | 40 | Rₚ |
| 3 | L3 | 37 | 56 | Sₚ |
| 4 | L4 | 62 | 26 | Rₚ |
| 5 | L5 | 38 | 34 | Sₚ |
| 6 | L6 | 39 | 58 | Rₚ |
| 7 | L7 | 48 | 41 | Sₚ |
| 8 | L8 | <5^{a} | N.D. | N.D. |
| 9 | L9 | <5^{a} | N.D. | N.D. |
| 10 | dcypf | 53 | N.D. | N.D. |

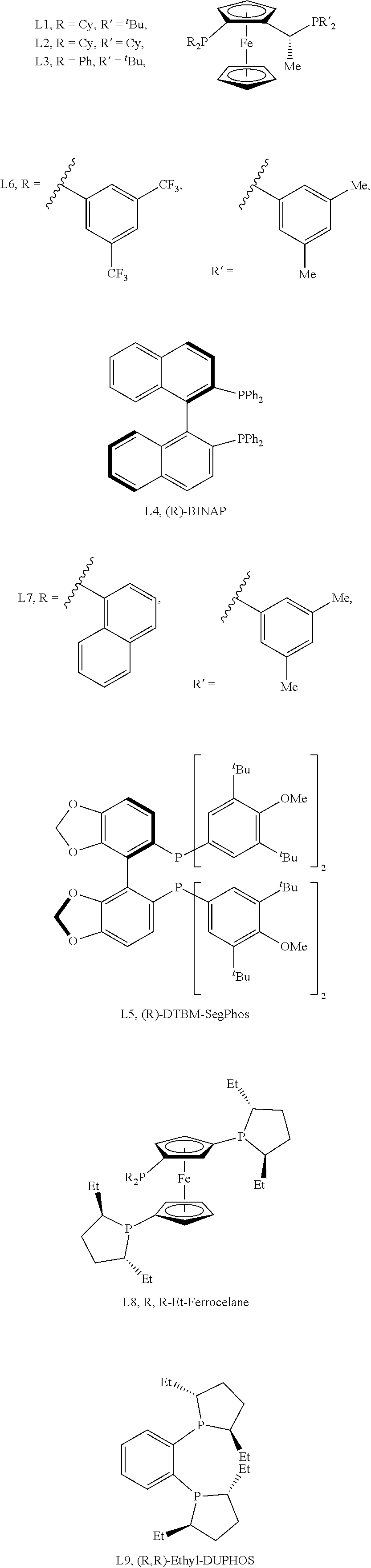

### G. Solvent Screen

A 20-mL vial was charged with a magnetic stir bar, and 3a (0.011 g, 0.025 mmol). The vial was brought into a glovebox, then Cs2CO3 (0.025 g, 0.075 mmol, 3.0 equiv), 1.00 mL of a corresponding solution of [(allyl)PdCl]2 (1.25 mM, 1.25 μmol, 5.0 mol %), L1 (0.028 g, 0.0050 mmol, 20.0 mol %), and a solvent (9.0 mL) were added. The vial was sealed and brought outside and placed in an aluminum heating block preheated at 60° C. The mixture was stirred (800 rpm) for 16 h. After cooling to room temperature, the mixture was filtered through a glass pipet packed with Celite (1×2 cm) using 5.0 mL of THF for washing. The filtrate was concentrated by rotary evaporator, and the residues were loaded onto a RediSep Gold® Normal-Phase Silica 4-gram column. The column was eluted using a Combiflash with a mixture of 30% EtOAc/hexanes for 3.0 CV (13 mL/minute). The product started to elute after 2.0 CV. The combined product-containing fractions were collected. The solvent was removed using rotary evaporator and the residue was further dried under high vacuum to give 4a as a colorless oil. Note: Cs2CO3 was ground with a pestle and mortar in the glovebox to a fine powder. It is advantageous to apply a stir rate of 800 rpm to the reactions to ensure reproducibility.

Scheme 7
Solvent Screening

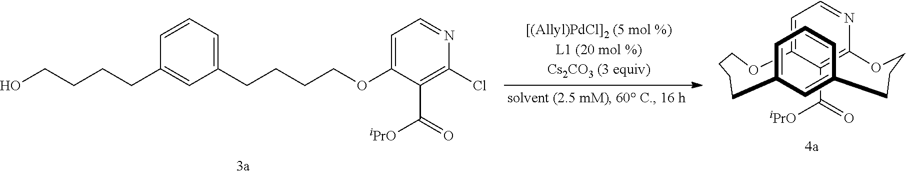

| Entry | Solvent | Yield (%) | ee (%) | Configuration |
|---|---|---|---|---|
| 1 | THF | 85 | 93 | Rₚ |
| 2 | dioxane | 86 | 87 | Rₚ |
| 3 | MeCN | 92 | 63 | Rₚ |
| 4 | DMF | 33 | 60 | Rₚ |
| 5 | toluene | 49 | 88 | Rₚ |
| 6 | DCM | <1 | N.D. | N.D. |

### H. Base and Base Loading Screen

A 20-mL vial was charged with a magnetic stir bar, and 3a (0.011 g, 0.025 mmol). The vial was brought into a glovebox, then a base (0.075 mmol, 3.0 equiv), 1.00 mL of a THF solution of [(allyl)PdCl]2 (1.25 mM, 1.25 μmol, 5.0 mol %), L1 (0.028 g, 0.0050 mmol, 20.0 mol %), and THF (9.0 mL) were added. The vial was sealed and brought outside and placed in an aluminum heating block preheated at 60° C. The mixture was stirred (800 rpm) for 16 h. After cooling to room temperature, the mixture was filtered through a glass pipet packed with Celite (1×2 cm) using 5.0 mL of THF for washing. The filtrate was concentrated by rotary evaporator, and the residues were loaded onto a RediSep Gold® Normal-Phase Silica 4-gram column. The column was eluted using a Combiflash with a mixture of 30% EtOAc/hexanes for 3.0 CV (13 mL/minute). The product started to elute after 2.0 CV. The combined product-containing fractions were collected. The solvent was removed using rotary evaporator and the residue was further dried under high vacuum to give 4a as a colorless oil. Note: Cs2CO3 was ground with a pestle and mortar in the glovebox to a fine powder. It is advantageous to apply a stir rate of 800 rpm to the reactions to ensure reproducibility.

Scheme 8
Base and base loading screen

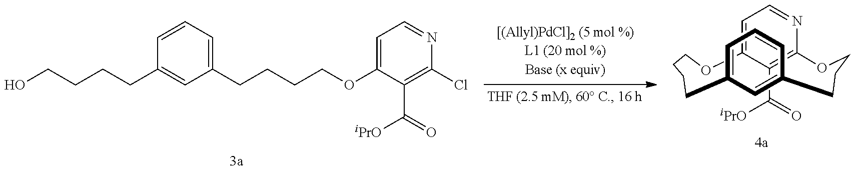

| Entry | Base | Base loading | Yield (%) | ee (%) | Configuration |
|---|---|---|---|---|---|
| 1 | Cs₂CO₃ | 3 equiv | 85 | 93 | Rₚ |
| 2 | Cs₂CO₃ | 5 equiv | 80 | 89 | Rₚ |
| 3 | Cs₂CO₃ | 10 equiv | 83 | 83 | Rₚ |
| 4 | K₂CO₃ | 3 equiv | 77 | 87 | Rₚ |
| 5 | K₃PO₄ | 3 equiv | 68 | 91 | Rₚ |
| 6 | KF | 3 equiv | <1 | N.D. | Rₚ |
| 7 | CsF | 3 equiv | 15 | 75 | Rₚ |
| 8 | KOAc | 3 equiv | 52 | 22 | Rₚ |
| 9 | NaO^{t}Bu | 3 equiv | 18 | 13 | Sₚ |

### I. Palladium Source Screen

A 20-mL vial was charged with a magnetic stir bar, and 3a (0.011 g, 0.025 mmol). The vial was brought into a glovebox, then Cs2CO3 (0.025 g, 0.075 mmol, 3.0 equiv), 1.00 mL of a THF solution of palladium catalyst (25 mM, 2.5 μmol, 10.0 mol %), L1 (0.028 g, 0.0050 mmol, 20.0 mol %), and THF (9.0 mL) were added. The vial was sealed and brought outside and placed in an aluminum heating block preheated at 60° C. The mixture was stirred (800 rpm) for 16 h. After cooling to room temperature, the mixture was filtered through a glass pipet packed with Celite (1×2 cm) using 5.0 mL of THF for washing. The filtrate was concentrated by rotary evaporator, and the residues were loaded onto a RediSep Gold® Normal-Phase Silica 4-gram column. The column was eluted using a Combiflash with a mixture of 30% EtOAc/hexanes for 3.0 CV (13 mL/minute). The product started to elute after 2.0 CV. The combined product-containing fractions were collected. The solvent was removed using rotary evaporator and the residue was further dried under high vacuum to give 4a as a colorless oil. Characterization data are provided in section 12. Note: Cs2CO3 was ground with a pestle and mortar in the glovebox to a fine powder. It is advantageous to apply a stir rate of 800 rpm to the reactions to ensure reproducibility.

Scheme 9
Palladium source screen.

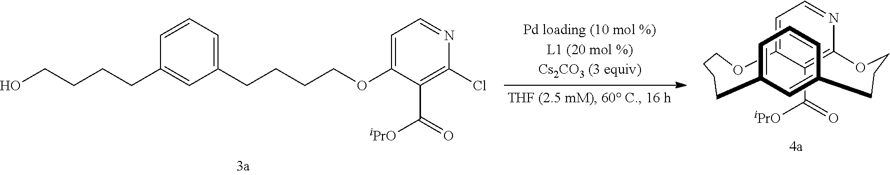

| Entry | Pd source | Yield (%) | ee (%) | Configuration |
|---|---|---|---|---|
| 1 | [(Allyl)PdCl]₂ | 85 | 93 | Rₚ |
| 2 | [(n-Butenyl)PdCl]₂ | 87 | 91 | Rₚ |
| 3 | [(π-cinnamyl)PdCl]₂ | 95 | 90 | Rₚ |
| 4 | Pd₂(dba)₃ | <1 | N.D. | N.D. |
| 5 | Bis(benzonitrile)PdCl₂ | 82 | 86 | Rₚ |
| 6 | Pd(OAc)₂ | 62 | 88 | Rₚ |
| 7 | Pd(PPh₃)₄ | 80 | 86 | Rₚ |

### J. Concentration Screening

An oven-dried 100-mL Schlenk flask was charged with a magnetic stir bar. The flask was brought into a glovebox, then Cs2CO3 (0.098 g, 0.30 mmol, 3.0 equiv), [(allyl) PdCI]2 (0.0018 g, 0.005 mmol, 5.0 mol %), L1 (0.011 g, 0.020 mmol, 20.0 mol %), THF (40, 20 or 5 mL), and 1.00 mL of a solution of 3a (0.10 M, 0.10 mmol) were added. The flask was sealed and brought outside and placed in an oil bath preheated at 60° C. The mixture was stirred (800 rpm) for 16 h. After cooling to room temperature, the mixture was filtered through a pad of Celite (1.0 g). The filtrate was concentrated by rotary evaporator, and the residues were loaded onto a RediSep Gold® Normal-Phase Silica 4-gram column. The column was eluted using a Combiflash with a mixture of 30% EtOAc/hexanes for 3.0 CV (13 mL/minute). The product started to elute after 2.0 CV. The combined product-containing fractions were collected. The solvent was removed using rotary evaporator and the residue was further dried under high vacuum to give 4a as a colorless oil. Characterization data are provided in section 12. Note: Cs2CO3 was ground with a pestle and mortar in the glovebox to a fine powder. It is advantageous to apply a stir rate of 800 rpm to the reactions to ensure reproducibility.

Scheme 10
Concentration screening.

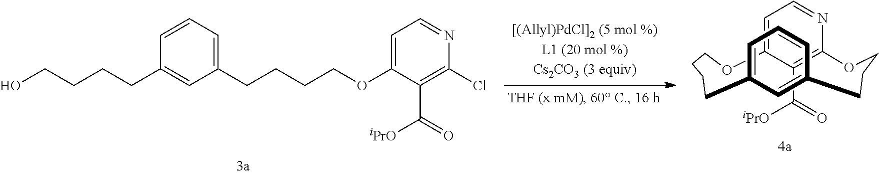

| Entry | Concentration | Yield (%) | ee (%) | Configuration |
|---|---|---|---|---|
| 1 | 2.5 mM | 85 | 87 | Rₚ |
| 2 | 5.0 mM | 73 | 88 | Rₚ |
| 3 | 20.0 mM | 72 | 85 | Rₚ |

### K. Temperature Screen

A 20-mL vial was charged with a magnetic stir bar, and 3a (0.011 g, 0.025 mmol). The vial was brought into a glovebox, then Cs2CO3 (0.025 g, 0.075 mmol, 3.0 equiv), 1.00 mL of a THF solution of [(allyl)PdCl]2 (1.25 mM, 1.25 μmol, 5.0 mol %), L1 (0.028 g, 0.0050 mmol, 20.0 mol %), and THF (9.0 mL) were added. The vial was sealed and brought outside and placed in an aluminum heating block preheated at 40, 60, 80, or 100° C. The mixture was stirred (800 rpm) for 16 h. After cooling to room temperature, the mixture was filtered through a glass pipet packed with Celite (1×2 cm) using 5.0 mL of THF for washing. The filtrate was concentrated by rotary evaporator, and the residues were loaded onto a RediSep Gold® Normal-Phase Silica 4-gram column. The column was eluted using a Combiflash with a mixture of 30% EtOAc/hexanes for 3.0 CV (13 mL/minute). The product started to elute after 2.0 CV. The combined product-containing fractions were collected. The solvent was removed using rotary evaporator and the residue was further dried under high vacuum to give 4a as a colorless oil. Characterization data are provided in section 12. Note: Cs2CO3 was ground with a pestle and mortar in the glovebox to a fine powder. It is advantageous to apply a stir rate of 800 rpm to the reactions to ensure reproducibility.

Scheme 11
Temperature screening.

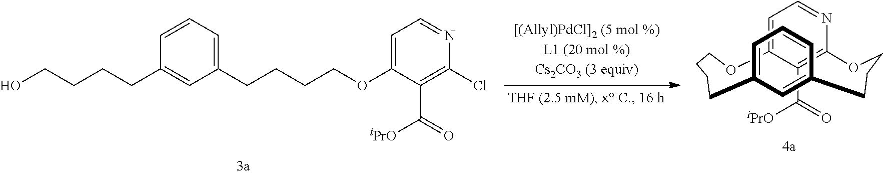

| Entry | Temperature | Yield (%) | ee (%) | Configuration |
|---|---|---|---|---|
| 1 | 40° C. | 31 | 75 | Rₚ |
| 2 | 60° C. | 85 | 93 | Rₚ |
| 3 | 80° C. | 83 | 90 | Rₚ |
| 4 | 100° C. | 76 | 85 | Rₚ |

### L. Synthesis of Example Macrocyclic Cyclophanes

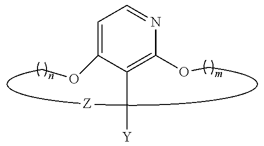

An oven-dried 100-mL Schlenk flask was charged with a magnetic stir bar. The flask was brought into a glovebox, then Cs2CO3 (0.098 g, 0.30 mmol), [(allyl)PdCl]2 (1.8 mg, 0.0050 mmol, 5.0 mol %), L1 (0.011 g, 0.020 mmol, 20.0 mol %), THF (39.0 mL), and 1.00 mL of a solution of 3(0.10 M, 0.10 mmol) were added. The flask was sealed and brought outside and placed in an oil bath preheated at 60° C. The mixture was stirred (800 rpm) for 16 h. After cooling to room temperature, the mixture was filtered through a pad of Celite (1.0 g).The filtrate was concentrated by rotary evaporator, and the residues were loaded onto a RediSep Gold® Normal-Phase Silica 12-gram column. The column was eluted using a Combiflash with a mixture of 30% EtOAc/hexanes for 6.0 CV (30 mL/minute). The product containing fractions was collected. The residue was dried with a high-vacuum oil pump to give 4. Note: Cs2CO3 was ground with a pestle and mortar in the glovebox to a fine powder. It is advantageous to apply 800 rpm stir rate to the reactions.

isopropyl 2,12-dioxa-1 (2,4)-pyridina-7 (1,3)-benzenacyclododecaphane-13-carboxylate (4a): Following general procedure D, compound 4a was isolated as a white solid (32.6 mg, 0.85 mmol, 85% yield, 87% ee) from the reaction of 3a (42.0 mg, 0.100 mmol).

cyclopentyl 2,12-dioxa-1 (2,4)-pyridina-7 (1,3)-benzenacyclododecaphane-13- -carboxylate (4b): Following general procedure D, compound 4b was isolated as a colorless oil (32mg, 0.079 mmol, 79% yield, 92% ee) from the reaction of 3b (44.6 mg, 0.100 mmol).

cyclohexyl 2,12-dioxa-1 (2,4)-pyridina-7 (1,3)-benzenacyclododecaphane-13- -carboxylate (4c): Following general procedure D, compound 4c was isolated as a colorless oil (30.0mg, 0.070 mmol, 70% yield, 88% ee) from the reaction of 3c (46.0 mg, 0.100 mmol).

ethyl 2,12-dioxa-1 (2,4)-pyridina-7 (1,3)-benzenacyclododecaphane-13- -carboxylate (4d): Following general procedure D, compound 4d was isolated as a colorless oil (24.2mg, 0.065 mmol, 65% yield, 89% ee) from the reaction of 3d (40.6 mg, 0.100 mmol).

butyl 2,12-dioxa-1 (2,4)-pyridina-7 (1,3)-benzenacyclododecaphane-13- -carboxylate (4e): Following general procedure D, compound 4e was isolated as a colorless oil (27.7mg, 0.069 mmol, 69% yield, 87% ee) from the reaction of 3e (43.4 mg, 0.100 mmol).

isopropyl 2,10-dioxa-1 (2,4)-pyridina-6 (1,3)-benzenacyclodecaphane-13- -carboperoxoate (4f): Following general procedure D, compound 4f was isolated as a white solid (28.6mg, 0.077 mmol, 77% yield, 84% ee) from the reaction of 3f (39.2 mg, 0.100 mmol).

isopropyl 2,6,8,12-tetraoxa-1 (2,4)-pyridina-7 (1,3)-benzenacyclododecaphane-13- -carboxylate (4g): Following general procedure D, compound 4g was isolated as a white solid (22.8mg, 0.059 mmol, 59% yield, 79% ee) from the reaction of 3g (42.4 mg, 0.100 mmol).

isopropyl 2,12-dioxa-1 (2,4)-pyridina-7 (1,2)-benzenacyclododecaphane-13- -carboxylate (4h): Following general procedure D, compound 4h was isolated as a white solid (29.1mg, 0.076 mmol, 76% yield, 79% ee) from the reaction of 3h (42.0 mg, 0.100 mmol).

isopropyl 2,12-dioxa-1 (2,4)-pyridina-7 (1,4)-benzenacyclododecaphane-13- -carboxylate (4i): Following general procedure D, compound 4i was isolated as a white solid (23.4 mg, 0.061 mmol, 61% yield, 83% ee) from the reaction of 3i (42.0 mg, 0.100 mmol).

isopropyl 2,11-dioxa-1 (2,4)-pyridinacycloundecaphane-13-carboxylate (4j): Following general procedure D, compound 4j was isolated as a colorless oil (19.6 mg, 0.064 mmol, 64% yield, 83% ee) from the reaction of 3j (34.4 mg, 0.100 mmol).

isopropyl 2,12-dioxa-1 (2,4)-pyridinacyclododecaphane-13-carboxylate (4k): Following general procedure D, compound 4k was isolated as a colorless oil (22.1 mg, 0.069 mmol, 69% yield, 90% ee) from the reaction of 3k (35.8 mg, 0.100 mmol).

isopropyl 2,13-dioxa-1 (2,4)-pyridinacyclotridecaphane-13-carboxylate (41): Following general procedure D, compound 41 was isolated as a colorless oil (23.8 mg, 0.071 mmol, 71% yield, 88% ee) from the reaction of 31 (37.2 mg, 0.100 mmol).

isopropyl 2,14-dioxa-1 (2,4)-pyridinacyclotetradecaphane-13-carboxylate (4m): Following general procedure D, compound 4m was isolated as a colorless oil (26.9 mg, 0.077 mmol, 77% yield, 89% ee) from the reaction of 3m (38.6 mg, 0.100 mmol).

isopropyl 2,15-dioxa-1 (2,4)-pyridinacyclopentadecaphane-13-carboxylate (4n): Following general procedure D, compound 4n was isolated as a colorless oil (25.0 mg, 0.069 mmol, 69% yield, 85% ee) from the reaction of 3n (40.0 mg, 0.100 mmol).

2,10-dioxa-1 (2,4)-pyridina-6 (1,3)-benzenacyclodecaphane (4q): Following the procedure in 11.1, compound 4q was isolated as a colorless oil (5.9 mg, 0.022 mmol, 22% yield) from the reaction of 1f (14.8 mg, 0.100 mmol) and 2b (19.4 mg, 0.100 mmol).

### M. Macrocyclization to Produce (Sp)-Enantiomers

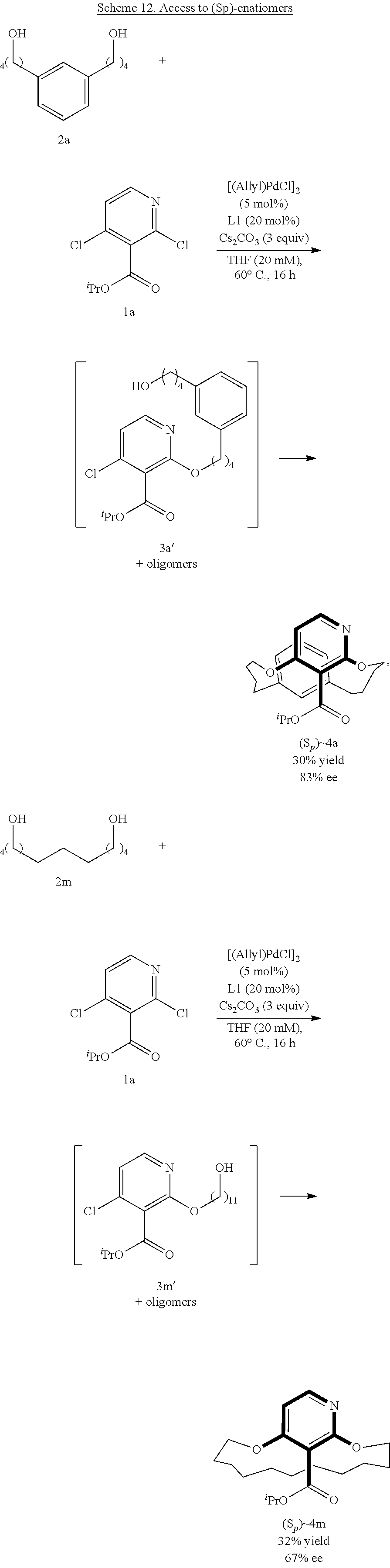

An oven-heated 20-mL vial was charged with a magnetic stir bar. 1a (0.022 g, 0.10 mmol, 1.0 equiv) and corresponding diol 2a or 2m (0.10 mmol, 1.0 equiv) were added into the vial. The vial was brought into a glovebox, then Cs2CO3 (0.098 g, 0.30 mmol), [(allyl)PdCl]2 (0.0018 g, 0.0050 mmol, 5.0 mol %), L1 (0.011 g, 0.020 mmol, 20.0 mol %), and THF (5.0 mL) were added. The vial was sealed and brought outside and placed in a heating block preheated at 60° C. The mixture was stirred at 800 rpm. NMR samples were made by removing 0.10 mL of the reaction mixture at 2 h and at 20 h from the same reaction vial. The solvent was removed by rotary evaporator and the residue was dissolved in CDCl3. 1H NMR spectra were then taken at ambient temperature. After cooling to room temperature, the remaining reaction mixture was filtered through a funnel with Celite (1.0 g). The filtrate was concentrated by rotary evaporator, and the residues were loaded onto a RediSep Gold® Normal-Phase Silica 12-gram column. The column was eluted using a Combiflash with a mixture of 20% EtOAc/hexanes for 5.0 minutes (30 mL/minute). The product was analyzed by chiral HPLC. Note: Cs2CO3 was ground with a pestle and mortar in the glovebox to a fine powder. It is advantageous to apply 800 rpm stir rate to the reactions.

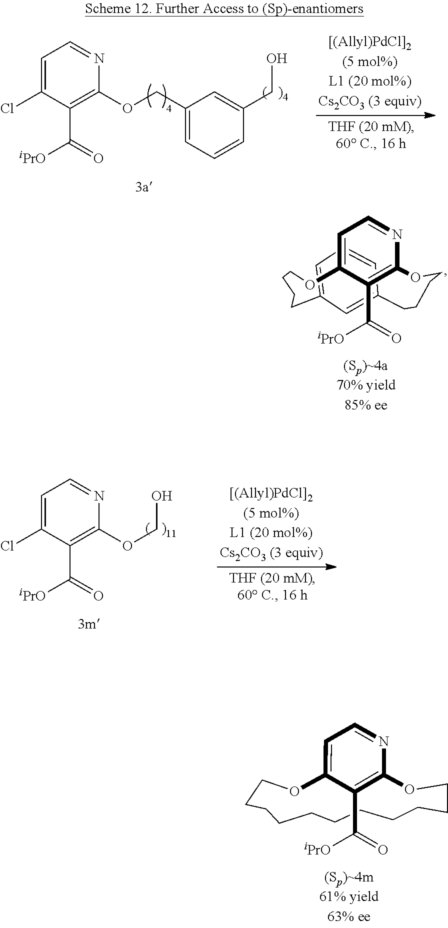

An oven-dried 100-mL Schlenk flask was charged with a magnetic stir bar. The flask was brought into a glovebox, then Cs2CO3 (0.098 g, 0.30 mmol), [(allyl)PdCl]2 (1.8 mg, 0.0050 mmol, 5.0 mol %), L1 (0.011 g, 0.020 mmol, 20.0 mol %), THF (4.0 mL), and 1.00 mL of a solution of 3a′ or 3m′ (0.10 M, 0.10 mmol) were added. The flask was sealed and brought outside and placed in an oil bath preheated at 60° C. The mixture was stirred at 800 rpm for 16 h. After cooling to room temperature, the mixture was filtered through a pad of Celite (1.0 g). The filtrate was concentrated by rotary evaporator, and the residues were loaded onto a RediSep Gold® Normal-Phase Silica 12-gram column. The column was eluted using a Combiflash with a mixture of 30% EtOAc/hexanes for 6.0 CV (30 mL/minute). The product containing fractions was collected. The residue was dried with a high-vacuum oil pump to give 4. Note: Cs2CO3 was ground with a pestle and mortar in the glovebox to a fine powder. It is advantageous to apply 800 rpm stir rate to the reactions.

### N. Summary

These results illustrate achievement of enantioselective Pd-catalyzed C—O bond-forming macrocyclizations and access to structurally diverse planar chiral metacyclophanes in good yields and enantioselectivities. Additionally, two enantiomers of the metacyclophane can be obtained from two regioisomeric linear precursors using the same enantiomer of the chiral ligand. Lastly, anti and syn conformational preferences of metacyclophanes can be exploited for selected stereoisomer formation, providing further opportunities for the rational design and facile synthesis of new planar chiral macrocyclic metacyclophanes with conformational control for applications in catalysis and medicinal chemistry.

### Additional Embodiments

The following exemplary embodiments are provided, the numbering of which is not to be construed as designating levels of importance:

Embodiment 1 provides a method of synthesizing a chiral metacyclophane, comprising contacting a substrate with a palladium cross-coupling catalyst, base, and a chiral ligand, wherein the substrate has a structure according to Formula I:

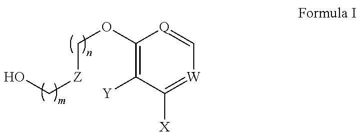

wherein Q is CH or N; W is CH or N; X is a halide; Y is a sterically bulky group; m is 0 to 10; n is 0 to 10; and Z is a divalent linking group.

Embodiment 2 provides the method of Embodiment 1, wherein at least one of Q and W is N.

Embodiment 3 provides the method of Embodiments 1 to 2, wherein Q is N and W is CH.

Embodiment 4 provides the method of Embodiments 1 to 3, wherein Q is CH and W is N.

Embodiment 5 provides the method of Embodiments 1 to 4, wherein Z is a divalent aromatic group.

Embodiment 6 provides the method of Embodiments 1 to 5, wherein Z is an optionally substituted phenylene linker.

Embodiment 7 provides the method of Embodiments 1 to 6, wherein Z is an optionally substituted divalent alkyl group.

Embodiment 8 provides the method of Embodiments 1 to 7, wherein Z is an optionally substituted C3-C8 alkyl linker.

Embodiment 9 provides the method of Embodiments 1 to 8, wherein Z provides a linear backbone of at least three atoms between points of attachment.

Embodiment 10 provides the method of Embodiments 1 to 9, wherein n, m, and Z together provide a linear backbone of at least 8 atoms between points of attachment.

Embodiment 11 provides the method of Embodiments 1 to 10, wherein Z comprises an aromatic group.

Embodiment 12 provides the method of Embodiments 1 to 11, wherein Y comprises a branched C3-C10 alkyl, cycloalkyl, aryl, heteroaryl, or non-aromatic heterocyclyl.

Embodiment 13 provides the method of Embodiments 1 to 12, wherein Y has the structure:

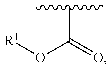

wherein R1 is an alkyl, cycloalkyl, aryl, heteroaryl, or non-aromatic heterocyclyl.

Embodiment 14 provides the method of Embodiments 1 to 13, wherein Y has the structure:

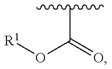

wherein R1 is a branched C3-C6 alkyl group.

Embodiment 15 provides the method of Embodiments 1 to 14, wherein the palladium cross-coupling catalyst is[(Allyl)PdCl]2.

Embodiment 16 provides the method of Embodiments 1 to 15, wherein the palladium cross-coupling catalyst is utilized at approximately 0.1 mol % to approximately 50 mol %.

Embodiment 17 provides the method of Embodiments 1 to 16, wherein the palladium cross-coupling catalyst is utilized at approximately 10 mol %.

The method of claim 1, when the palladium cross-coupling catalyst, base, chiral ligand, and substrate are stirred with a magnetic stirrer or mechanical stirrer at an RPM of at least 500.

Embodiment 18 provides the method of Embodiments 1 to 17, wherein the chiral ligand is a diphosphine ligand.

Embodiment 19 provides the method of Embodiments 1 to 18, wherein the chiral ligand contains an aromatic moiety.

Embodiment 20 provides the method of Embodiments 1 to 19, wherein the chiral ligand is a ferrocene-containing ligand.

Embodiment 21 provides the method of Embodiments 1 to 20, wherein the chiral ligand is a JosiPhos-type ligand.

Embodiment 22 provides the method of Embodiments 1 to 21, wherein the chiral ligand is utilized at approximately 1:2 Pd/ligand ratio to 1:5 Pd/Ligand Ratio.

Embodiment 23 provides the method of Embodiments 1 to 22, wherein the base is Cs2CO3, NaOtBu, CsF, or KOAc.

Embodiment 24 provides the method of Embodiments 1 to 23, wherein the base is an inorganic carbonate.

Embodiment 25 provides the method of Embodiments 1 to 24, wherein the base is utilized at about 2 to about 5 equivalents relative to the substrate.

Embodiment 26 provides the method of Embodiments 1 to 25, wherein the palladium cross-coupling catalyst, base, chiral ligand, and substrate are heated to a temperature of about 50° C. or higher.

Embodiment 27 provides a compound having a structure according to Formula II, or a salt thereof:

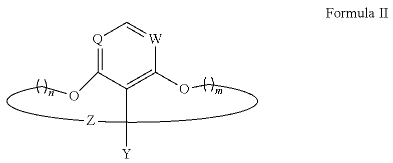

wherein Q is CH or N; W is CH or N; Y is a sterically bulky group; m is 0 to 10; n is 0 to 10; and Z is a divalent linking group.

Embodiment 28 provides a compound of Embodiment 27, wherein at least one of Q and W is N.

Embodiment 29 provides a compound of any one of Embodiments 27-28, wherein Q is N and W is CH.

Embodiment 30 provides a compound of any one of Embodiments 27-29, wherein Q is CH and W is N.

Embodiment 31 provides a compound of any one of Embodiments 27-30, wherein Z is a divalent aromatic group.

Embodiment 32 provides a compound of any one of Embodiments 27-31, wherein Z is an optionally substituted phenylene linker.

Embodiment 33 provides a compound of any one of Embodiments 27-32, wherein Z is an optionally substituted divalent alkyl group.

Embodiment 34 provides a compound of any one of Embodiments 27-33, wherein Z is an optionally substituted C3-C8 alkyl linker.

Embodiment 35 provides a compound of any one of Embodiments 27-34, wherein Z provides a linear backbone of at least three atoms between points of attachment.

Embodiment 36 provides a compound of any one of Embodiments 27-35, wherein n, m, and Z together provide a linear backbone of at least 8 atoms between points of attachment.

Embodiment 37 provides a compound of any one of Embodiments 27-36, wherein Z comprises an aromatic group.

Embodiment 38 provides a compound of any one of Embodiments 27-37, wherein Y comprises a branched C3-C10 alkyl, cycloalkyl, aryl, heteroaryl, or non-aromatic heterocyclyl

Embodiment 39 provides a compound of any one of Embodiments 27-38, wherein Y has the structure:

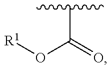

wherein R1 is an alkyl, cycloalkyl, aryl, heteroaryl, or non-aromatic heterocyclyl.

Embodiment 40 provides a compound of any one of Embodiments 27-39, wherein Y has the structure:

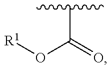

wherein R1 is a branched C3-C6 alkyl group.

Embodiment 41 provides a compound of any one of Embodiments 27-40, having a structure according to Formula III, or a salt thereof:

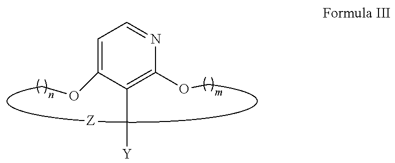

Y is a sterically bulky group; m is 0 to 10; n is 0 to 10; and Z is a divalent linking group.

Embodiment 42 provides a compound of any one of Embodiments 27-41, having the structure:

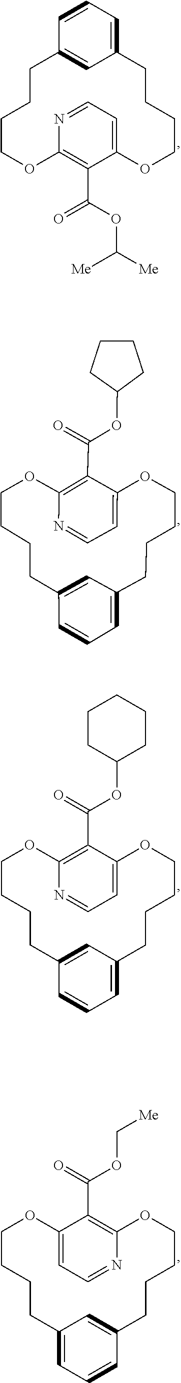 [SMILES: CC(C)OC(=O)c1c2ccnc1OCCCCc1cccc(c1)CCCCO2 ; O=C(OC1CCCC1)c1c2ccnc1OCCCCc1cccc(c1)CCCCO2 ; O=C(OC1CCCCC1)c1c2ccnc1OCCCCc1cccc(c1)CCCCO2 ; CCOC(=O)c1c2ccnc1OCCCCc1cccc(c1)CCCCO2]

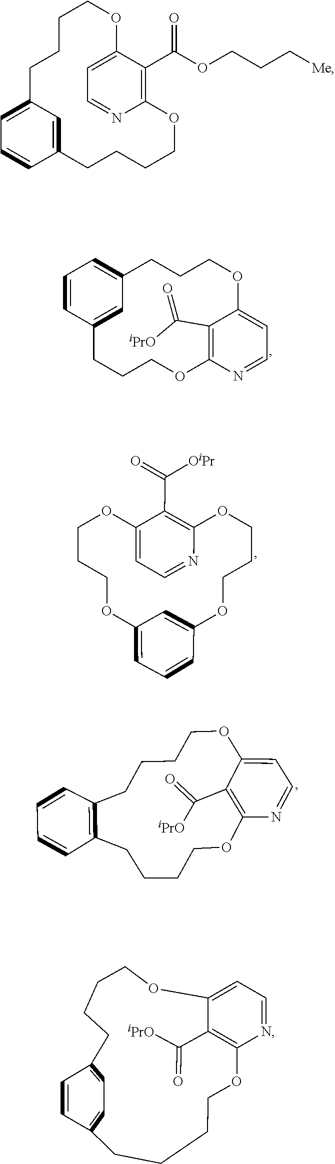 [SMILES: CCCCOC(=O)c1c2ccnc1OCCCCc1cccc(c1)CCCCO2 ; CC(C)OC(=O)c1c2ccnc1OCCCc1cccc(c1)CCCO2 ; CC(C)OC(=O)c1c2ccnc1OCCCOc1cccc(c1)OCCCO2 ; CC(C)OC(=O)c1c2ccnc1OCCCCc1ccccc1CCCCO2 ; CC(C)OC(=O)c1c2ccnc1OCCCCc1ccc(cc1)CCCCO2]

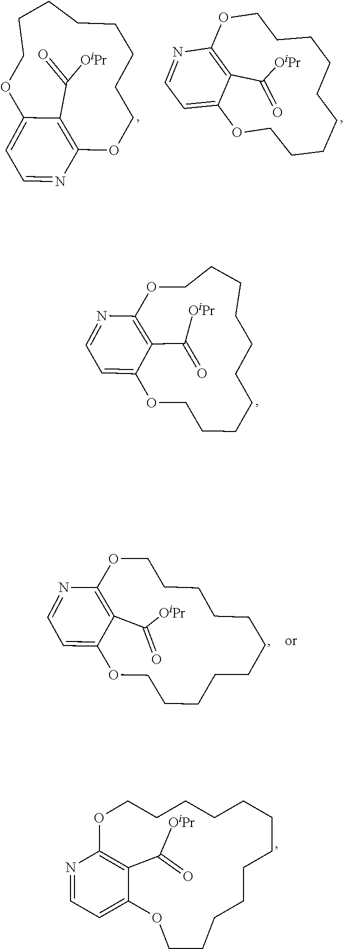

wherein the structure is a racemate, a non-racemic mixture of stereoisomers, an (Rp)-enantiomer, or an (Sp)-enantiomer.

Embodiment 43 provides a compound of any one of Embodiments 27-42, which is an (Sp)-enantiomer.

Embodiment 44 provides a compound of any one of Embodiments 27-43, which is an (Rp)-enantiomer.

Embodiment 45 provides a compound of any one of Embodiments 27-44, which has a syn-conformation.

Embodiment 46 provides a compound of any one of Embodiments 27-45, which has an anti-conformation.

Embodiment 47 provides a compound having the structure:

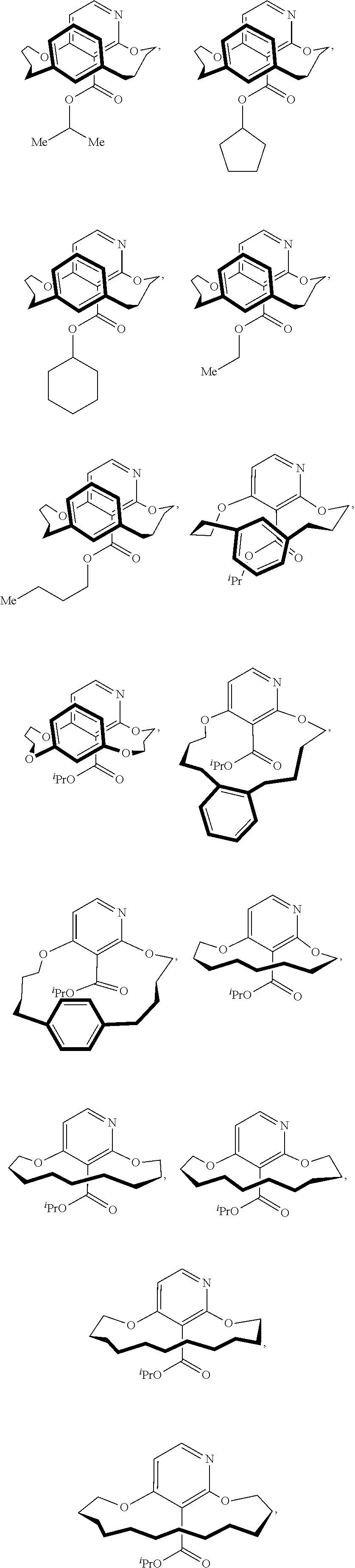

as an (Rp)-enantiomer, or a salt thereof.

Embodiment 48 provides a compound according to the description and figures herein.

Embodiment 49 provides a method according to the description and figures herein.

Embodiment 50 provides a compound or method of any one or any combination of Embodiments 1-47 optionally configured such that all elements or options recited are available to use or select from.

1. A method of synthesizing a chiral metacyclophane, comprising contacting a substrate with a palladium cross-coupling catalyst, base, and a chiral ligand, wherein the substrate has a structure according to Formula I: 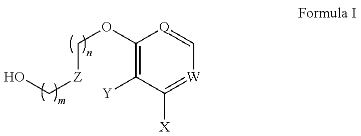
wherein Q is CH or N; W is CH or N; X is a halide; Y is a sterically bulky group; m is 0 to 10;
n is 0 to 10; and Z is a divalent linking group.

2. The method of claim 1, wherein at least one of Q and W is N.

3. (canceled)

4. (canceled)

5. (canceled)

6. (canceled)

7. (canceled)

8. (canceled)

9. The method of claim 1, wherein Z provides a linear backbone of at least three atoms between points of attachment.

10. The method of claim 1, wherein n, m, and Z together provide a linear backbone of at least 8 atoms between points of attachment.

11. The method of claim 1, wherein Z comprises an aromatic group.

12. The method of claim 1, wherein Y comprises a branched C3-C10 alkyl, cycloalkyl, aryl, heteroaryl, or non-aromatic heterocyclyl.

13. The method of claim 1, wherein Y has the structure: 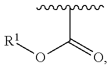
wherein R1 is an alkyl, cycloalkyl, aryl, heteroaryl, or non-aromatic heterocyclyl.

14. (canceled)

15. (canceled)

16. The method of claim 1, wherein the palladium cross-coupling catalyst is utilized at approximately 0.1 mol % to approximately 50 mol %.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. A compound having a structure according to Formula II, or a salt thereof: 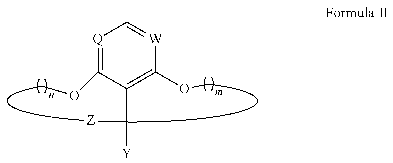
wherein Q is CH or N; W is CH or N; Y is a sterically bulky group; m is 0 to 10; n is 0 to 10;
and Z is a divalent linking group.

29. The compound of claim 28, wherein at least one of Q and W is N.

30. (canceled)

31. (canceled)

32. (canceled)

33. (canceled)

34. (canceled)

35. (canceled)

36. The compound of claim 28, wherein Z provides a linear backbone of at least three atoms between points of attachment.

37. The compound of claim 28, wherein n, m, and Z together provide a linear backbone of at least 8 atoms between points of attachment.

38. The compound of claim 28, wherein Z comprises an aromatic group.

39. The compound of claim 28, wherein Y comprises a branched C3-C10 alkyl, cycloalkyl, aryl, heteroaryl, or non-aromatic heterocyclyl.

40. The compound of claim 28, wherein Y has the structure: 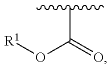
wherein R1 is an alkyl, cycloalkyl, aryl, heteroaryl, or non-aromatic heterocyclyl.

41. (canceled)

42. The compound of claim 28, having a structure according to Formula III, or a salt thereof: 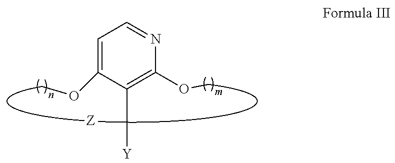
Y is a sterically bulky group; m is 0 to 10; n is 0 to 10; and Z is a divalent linking group.

43. A compound having the structure: 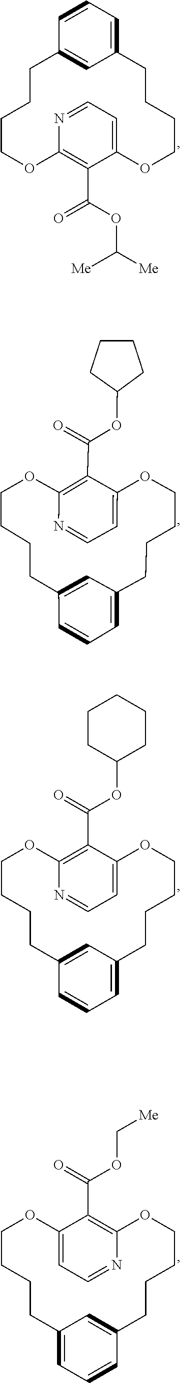 [SMILES: CC(C)OC(=O)c1c2ccnc1OCCCCc1cccc(c1)CCCCO2 ; O=C(OC1CCCC1)c1c2ccnc1OCCCCc1cccc(c1)CCCCO2 ; O=C(OC1CCCCC1)c1c2ccnc1OCCCCc1cccc(c1)CCCCO2 ; CCOC(=O)c1c2ccnc1OCCCCc1cccc(c1)CCCCO2] 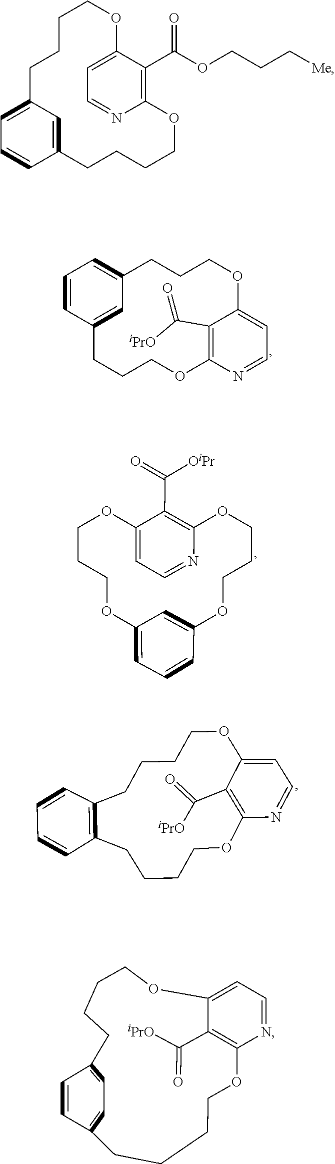 [SMILES: CCCCOC(=O)c1c2ccnc1OCCCCc1cccc(c1)CCCCO2 ; CC(C)OC(=O)c1c2ccnc1OCCCc1cccc(c1)CCCO2 ; CC(C)OC(=O)c1c2ccnc1OCCCOc1cccc(c1)OCCCO2 ; CC(C)OC(=O)c1c2ccnc1OCCCCc1ccccc1CCCCO2 ; CC(C)OC(=O)c1c2ccnc1OCCCCc1ccc(cc1)CCCCO2] 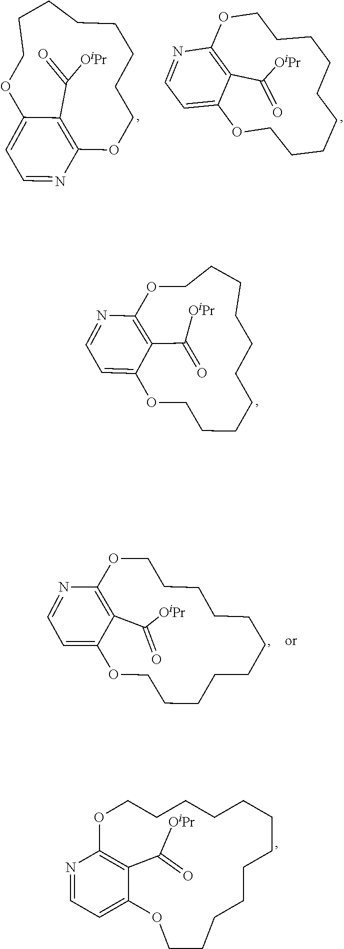
wherein the structure is a racemate, a non-racemic mixture of stereoisomers, an (Rp)-enantiomer, or an (Sp)-enantiomer.

44. (canceled)

45. (canceled)

46. The compound of claim 28, which has a syn-conformation.

47. The compound of claim 28, which has an anti-conformation.

48. The compound of claim 43 having the structure: 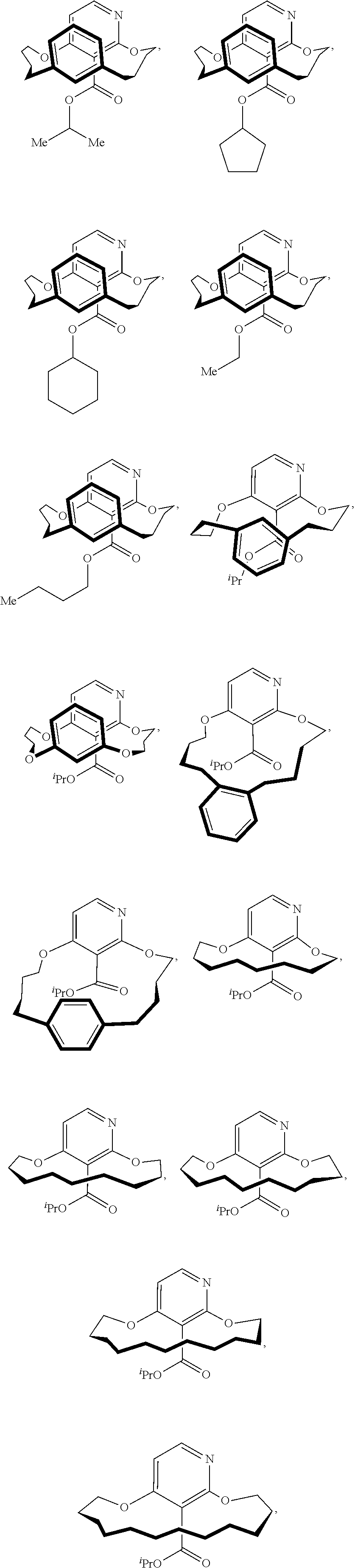
as an (Rp)-enantiomer, or a salt thereof.
